(19) United States
(12) Patent Application Publication
(10) Pub. No.: US 2025/0297272 A1
(43) Pub. Date: Sep. 25, 2025

(54) METHODS AND COMPOSITIONS FOR PRODUCING PLANTS HAVING HIGH VEGETATIVE FATTY ACIDS

(71) Applicant: NUTECH VENTURES, Lincoln, NE (US)

(72) Inventors: Edgar CAHOON, Lincoln, NE (US); Kiyoul PARK, Lincoln, NE (US); Truyen QUACH, Lincoln, NE (US); Thomas CLEMENTE, Lincoln, NE (US)

(21) Appl. No.: 18/853,775

(22) PCT Filed: Apr. 5, 2023
(86) PCT No.: PCT/US2023/065391

§ 371 Date: Oct. 3, 2024

Related U.S. Application Data
(60) Provisional application No. 63/327,704, filed on Apr. 5, 2022.

(51) Int. Cl. C12N 15/82; C07K 14/415; C12N 9/10; C12N 9/16

(52) U.S. Cl. CPC C12N 15/8247; C07K 14/415; C12N 9/1029; C12N 9/16; C12Y 203/0102; C12Y 203/01051; C12Y 301/02014

(57) ABSTRACT

This disclosure describes transgenic plants engineered for enhanced amounts of fatty acids and triacylglycerols (TAGs) in vegetative tissues. This document describes novel approaches to develop biomass crops, such as Sorghum, for production of vegetative sources of vegetable oils in leaves and stems as alternative renewable diesel and sustainable aviation fuel (SAF) feedstocks. A new approach is described for engineering high vegetative oil production that involves expression of variant medium-chain fatty acid acyl-acyl carrier protein (AGP) thioesterases to drive vegetable oil production. This approach has been shown to be effective with two different acyl-ACP thioesterases, and the high oil production is maintained over multiple genetic generations in greenhouse and field cultivation systems.

### CROSS REFERENCE TO RELATED APPLICATIONS

This application claims the benefit of priority under 35 U.S.C. § 119(e) to U.S. Application No. 63/327,704 filed on Apr. 5, 2022, which is incorporated by reference in its entirety.

### FEDERALLY SPONSORED RESEARCH OR DEVELOPMENT

This invention was made with government support under DE-SC0018420 awarded by the U.S. Department of Energy. The government has certain rights in the invention.

### TECHNICAL FIELD

This disclosure generally relates to transgenic plants for making fatty acids and triacylglycerols in vegetative tissues.

### BACKGROUND

The rising global demand for renewable diesel and sustainable aviation fuel (SAF) necessitates the development of alternative vegetable oil feedstocks to supplement the current supply of oilseeds such as soybean and canola, oil palm, and fats from meat processing.

### SUMMARY

This document describes novel approaches to develop biomass crops, such as sorghum, for production of vegetative sources of vegetable oils in leaves and stems as alternative renewable diesel and sustainable aviation fuel (SAF) feedstocks. A new approach is described for engineering high vegetative oil production that involves expression of variant medium-chain fatty acid acyl-acyl carrier protein (ACP) thioesterases to drive vegetable oil production. This approach has been shown to be effective with two different acyl-ACP thioesterases, and the high oil production is maintained over multiple genetic generations in greenhouse and field cultivation systems. This approach is not only effective in conferring accumulation of triacylglycerols (TAG), the primary component of vegetable oils, but also yielded>four-fold increases in total fatty acids in stems of sweet and grain sorghum varieties.

In one aspect, nucleic acid constructs are provided that include at least one constitutive promoter driving expression of: a nucleic acid molecule encoding a diacylglycerol acyltransferase 1 (DGAT1) protein; a nucleic acid molecule encoding a Wrinkled1 (Wri1) protein; a nucleic acid molecule encoding an oleosin (Ole) protein; a nucleic acid molecule encoding a medium-chain acyl-ACP thioesterase; and a nucleic acid molecule encoding a lysophosphatidic acid acyltransferase 2 (LPAT2) protein.

In some embodiments, the DGAT protein is a Cuphea DGAT1 protein. In some embodiments, the wrinkled1 protein is a Sorghum wrinkled1 protein. In some embodiments, the oleosin protein is a sesame oleosin protein. In some embodiments, the medium-chain acyl-ACP thioesterase is a FatB protein. In some embodiments, the FatB protein is a Cuphea viscosissima FatB protein. In some embodiments, the medium-chain acyl-ACP thioesterase protein is a Cuphea palustris medium-chain acyl-ACP thioesterase protein. In some embodiments, the LPAT2 protein is a Cuphea viscosissima LPAT2 protein.

In another aspect, nucleic acid constructs are provided that include at least one constitutive promoter driving expression of: a nucleic acid molecule encoding a Cuphea avigera var. pulcherrima DGAT1 protein; a nucleic acid molecule encoding a sorghum Wrinkled1 protein; a nucleic acid molecule encoding a sesame oleosin protein; a nucleic acid molecule encoding a Cuphea viscosissima FatB protein; and a nucleic acid molecule encoding a Cuphea viscosissima LPAT2 protein.

In still another aspect, nucleic acid constructs are provided that include at least one constitutive promoter driving expression of: a nucleic acid molecule encoding a Cuphea avigera var. pulcherrima DGAT1 protein; a nucleic acid molecule encoding a sorghum Wrinkled1 protein; a nucleic acid molecule encoding a sesame oleosin protein; a nucleic acid molecule encoding a Cuphea palustris thioesterase protein; and a nucleic acid molecule encoding a Cuphea avigera var. pulcherrima LPATB protein.

In some embodiments, the nucleic acid molecule encoding the DGAT1 protein from Cuphea avigera var. pulcherrima has the nucleic acid sequence shown in SEQ ID NO:1. In some embodiments, the DGAT1 protein from Cuphea avigera var. pulcherrima has the amino acid sequence shown in SEQ ID NO:2.

In some embodiments, the nucleic acid molecule encoding the Wrinkled1 protein from sorghum has the nucleic acid sequence shown in SEQ ID NO:3. In some embodiments, the Wrinkled1 protein from sorghum has the amino acid sequence shown in SEQ ID NO:4.

In some embodiments, the nucleic acid molecule encoding the oleosin protein from sesame has the nucleic acid sequence shown in SEQ ID NO:5. In some embodiments, the oleosin protein from sesame has the amino acid sequence shown in SEQ ID NO:6.

In some embodiments, the nucleic acid molecule encoding the FatB protein from Cuphea viscosissima has the nucleic acid sequence shown in SEQ ID NO:7. In some embodiments, the FatB protein from Cuphea viscosissima has the amino acid sequence shown in SEQ ID NO:8.

In some embodiments, the nucleic acid molecule encoding the LPAT2 protein from Cuphea viscosissima has the nucleic acid sequence shown in SEQ ID NO:9. In some embodiments, the LPAT2 protein from Cuphea viscosissima has the amino acid sequence shown in SEQ ID NO:10.

In some embodiments, the nucleic acid molecule encoding the thioesterase protein from Cuphea palustris has the nucleic acid sequence shown in SEQ ID NO:13. In some embodiments, the thioesterase protein from Cuphea palustris has the amino acid sequence shown in SEQ ID NO:14.

In some embodiments, the nucleic acid molecule encoding the LPATB protein from Cuphea avigera var. pulcherrima has the nucleic acid sequence shown in SEQ ID NO:15. In some embodiments, the LPATB protein from Cuphea avigera var. pulcherrima has the amino acid sequence shown in SEQ ID NO:16.

In some embodiments, the at least one constitutive promoter is selected from the group consisting of a 35S promoter from cauliflower mosaic virus, a ubiquitin 1 promoter from maize, a ubiquitin 4 promoter from sugarcane, and a PGD1 promoter from rice. In some embodiments, the nucleic constructs described herein further include more than one constitutive promoter driving expression of the nucleic acid molecules. In some embodiments, the nucleic constructs described herein further include a plurality of constitutive promoters driving expression of the nucleic acid molecules.

In some embodiments, the nucleic constructs described herein further include at least one terminator sequence. In some embodiments, the nucleic constructs described herein, further include left border nucleic acid sequences and right border nucleic acid sequences. In some embodiments, the nucleic acid construct is a T-DNA.

In yet another aspect, methods of making a transgenic plant that produces increased amounts of fatty acids and/or triacylglycerols (TAGs) in vegetative tissues (relative to a corresponding plant lacking the construct) are provided. Such methods typically include transforming a plant cell with any of the nucleic acid constructs described herein, and regenerating the transformed plant cell into a plant, thereby making a plant that produces increased amounts of fatty acids and/or triacylglycerols (TAGs) in vegetative tissues relative to a corresponding plant lacking the construct.

In some embodiments, the plant is selected from the group consisting of sorghum, sugarcane, Miscanthus, maize, rye, and switch grass. In some embodiments, the transforming step is via Agrobacterium transformation. In some embodiments, the increased amounts of fatty acids in vegetative tissues is about two-fold, three-fold, or four-fold relative to the amount of fatty acids in vegetative tissues in a corresponding plant lacking the construct.

In one aspect, transgenic plants including any of the nucleic acid constructs described herein are provided. In some embodiments, the plant is selected from the group consisting of sorghum, sugarcane, Miscanthus, maize, rye, and switch grass. In some embodiments, the plant exhibits increased amounts of fatty acids and/or TAGs in vegetative tissues relative to a corresponding plant lacking the construct.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

FIG. 1 are schematic structures of T-DNA for generation of transgenic sorghum plants. LB; left border, RB; right border p35S; cauliflower mosaic virus 35S promoter, pUbi4; sugarcane ubiquitin4 promoter, pUbi1; maize ubiquitin1 promoter pPGD1; rice PGD1 promoter, pWR1; Wrinkled1-responsive promoter1, pWR2; Wrinkled1-responsive promoter2, t35S; cauliflower mosaic virus 35S terminator, tOcs; Agrobacterium Ocs terminator, tNos; Agrobacterium nopaline synthase (Nos) terminator, tPinII; potato PinII terminator, tOsACT; rice actin1 terminator, tSbACT; sorghum actin1 terminator.

FIG. 2 are graphs showing oil content in leaves and stalks from regenerated T₀ pPTN1569-expressing Tx430 background events. Upper panel shows structure of T-DNA that was introduced into sorghum. Iodine-stained TLC plates are shown in left panel. Red arrow shows TAG location on TLC plates. Error Bars mean±SD.

FIG. 3 are graphs showing oil increase in pPTN1586-expressing transgenic Ramada sorghum (To generation) plants. Upper panel shows structure of T-DNA that was introduced into sorghum. Iodine-stained TLC plates are shown in left panel. Red arrow shows TAG location in TLC. Error bars are mean±SD.

FIG. 4 are graphs showing the greenhouse experiment of Cuphea DGAT1-expressing transgenic T1 sorghum plants. Leaf TAG amounts were measured at the before-flowering stage. Data was obtained from (A) Tx430 background plants and (B) Ramada background transgenics. Error bar indicates mean±SD.

FIG. 5 are graphs showing phenotype analysis of a high oil sorghum event under the 2021 and 2022 Nebraska field summer conditions. Leaf and stem were collected at described developmental stages and analyzed using gas chromatography-flame ionization detection (GC-FID). Error bars represent ±SD (n≥5).

FIG. 6 are graphs showing biomass comparison between wild-type and a high oil sorghum event. Arial vegetative parts, including leaves and stems, of plants were cut and weighed for the fresh weight. Blue and orange represent wild-type and high oil sorghum (TZ424-5-3a), respectively. BF, before flowering; AF, after flowering; DAS, day after sowing. n=12. P-values from Student's t test are shown.

FIG. 7 is a graph showing the initial screening results of transgenic sorghum events. Leaf TAG content was measured from greenhouse-grown T₀ and T₁ events. Each point represents leaf TAG concentrations of individual plants.

FIG. 8 is a graph showing acyl-ACP thioesterase activity assay. Medium-chain fatty acid-specific thioesterase (FatB) activity was measured in the presence of 14:0-ACP. Enzyme reaction with 18:1A9-ACP is to measure common thioesterase (FatA) activity. Error bars represent ±SD (n=5). **P<0.01, Student's t test.

### DETAILED DESCRIPTION

This disclosure describes methods and compositions for enriching triacylglycerols (TAG) in vegetative tissues, including leaves and stems, of biomass crops. The methods include co-expressing a transgene for a specialized medium-chain fatty acid acyl-acyl carrier protein (ACP) thioesterase, with transgenes for enzymes and/or oil body-associated proteins. In this disclosure, transgenic expression of two distinct medium-chain acyl-ACP thioesterases were shown to yield 50 to 100-fold increase in TAG concentrations in sorghum leaves and stems when co-expressed with three transgenes related to TAG concentration in leaves and stems of non-engineered plants. In the absence of the medium-chain acyl-ACP thioesterases, expression of only the three transgenes yielded TAG concentrations ˜5-fold lower than those in leaves and stems of sorghum plants that co-express the medium-chain acyl-ACP thioesterases. These effects were observed over multiple generations in a greenhouse environment and two seasons of field production. As used herein, medium-chain thioesterases refer to enzymes in the FatB family of thioesterases that catalyze the release of fatty acid chains from acyl-carrier protein in de novo fatty acid synthesis using acyl-ACP substrates with fatty acid chains containing ≥8 and <16 carbon atoms. Representative medium-chain acyl-ACP thioesterases suitable for use in the compositions and methods described herein are Cuphea thioesterases and are shown in SEQ ID NOs:8 and 14, which are encoded by the nucleic acid sequences shown in SEQ ID NOs:7 and 13, respectively. Further examples of medium-chain acyl-ACP thioesterases suitable for use in the compositions and methods described herein include, without limitation, GenBank Accession Nos. U65643.1 (Myristica fragrans (nutmeg)); U65644.1 (Ulmus americana (elm)); AEM72519 (Cocos nucifera (coconut)); Q41635.1 (Umbellularia californica (California bay)); and CAD63310.1 (Lactbacilus plantaum WCFS1).

These studies include constitutive expression of transgenes for a Wrinkled 1 transcription factor, a diacylglycerol acyltransferase (DGAT (e.g., DGAT1, DGAT2)), and an oleosin. A representative wrinkled 1 sequence suitable for use in the compositions and methods described herein is a sorghum wrinkled 1 and is shown in SEQ ID NO:4, which is encoded by the nucleic acid sequence shown in SEQ ID NO:3. Further examples of wrinkled 1 sequences suitable for use in the compositions and methods described herein include, without limitation, GenBank Accession Nos. EU960249.1 (Zea mays); AJ575217 (Oryza sativa (rice)); MK138587 (Avena sativa (oat)); and NMV1_001035780 (Arabidopsis thaliana). Representative DGAT sequences suitable for use in the compositions and methods described herein include a Cuphea DGAT or an Arabidopsis DGAT having the sequences shown in SEQ ID NOs:2 or 12, which are encoded by the nucleic acid sequences shown in SEQ ID NOs:1 or 11, respectively. Further examples of DGAT sequences suitable for use in the compositions and methods described herein include, without limitation, GenBank Accession Nos. EU039830 (Zea mars (ZmDGA_T1-2)), KU744408 (Corylus americana); and XP_965438 (Neurospora crassa fungal DGAT2). A representative oleosin sequence suitable for use in the compositions and methods described herein is a sesame oleosin and is shown in SEQ ID NO:6, encoded by the nucleic acid shown in SEQ ID NO:5.

The particular challenge for engineering biomass crops is to block fatty acid and TAG catabolism through processes such as beta-oxidation, which naturally restrict the accumulation of fatty acids and TAG in plant vegetative tissues. The strategy described herein is unique because of the inclusion, or “stacking,” of medium-chain acyl-ACP thioesterases and an associated specialized lysophosphatidic acid acyltransferase (LPAT). Representative LPAT sequences suitable for use in the compositions and methods described herein include Cuphea LPAT sequences shown in SEQ ID NOs:10 and 16, which are encoded by the nucleic acid sequences shown in SEQ ID NOs:9 and 15, respectively.

While intended to produce vegetative TAG enriched in medium-chain fatty acids, this approach had the unexpected effect of strongly enhancing total fatty acid production and TAG accumulation in leaves and stems of two genetically distinct sorghum varieties. Leaves from plants at the T₀ generation for the top-performing events had increases in TAG concentrations that were two- to five-fold higher than increases conferred by only the “three-transgene combination” (i.e., the modified sorghum Wrinkled1 transcription factor, the Arabidopsis or Cuphea diacylglycerol acyltransferase (DGAT), and the sesame oleosin). This difference was more significant in the T₁ top-performing progeny. Leaf TAG concentrations in these events were up to nine-fold higher than increases conferred by only the “three-transgene combination.” As used herein, “oil” and “vegetable oil” can be used interchangeably and typically refer to fatty acids (e.g., a TAG-rich extract) from a plant material.

### Nucleic Acids and Polypeptides

As used herein, nucleic acids include DNA and RNA, and also can include one or more nucleotide analogs or backbone modifications. A nucleic acid can be single stranded or double stranded, and circular or linear.

A construct for expressing a nucleic acid (e.g., a nucleic acid that encodes a polypeptide) also is provided. Expression constructs are commercially available or can be produced by recombinant DNA techniques routine in the art. A construct containing a nucleic acid can have expression elements operably linked to such a nucleic acid, and further can include sequences such as those encoding a selectable marker (e.g., an antibiotic resistance gene). A construct containing a nucleic acid can encode a chimeric or fusion polypeptide (i.e., a polypeptide operatively linked to a heterologous polypeptide, which can be at either the N-terminus or C-terminus of the polypeptide). Representative heterologous polypeptides are those that can be used in purification of the encoded polypeptide (e.g., 6×His tag, glutathione S-transferase (GST))

Expression elements include nucleic acid sequences that direct and regulate expression of nucleic acid coding sequences. One example of an expression element is a promoter sequence. Expression elements also can include introns, enhancer sequences, response elements, or inducible elements that modulate expression of a nucleic acid. Expression elements can be of bacterial, yeast, insect, mammalian, or viral origin, and constructs can contain a combination of elements from different origins. As used herein, operably linked means that a promoter or other expression element(s) are positioned in a construct relative to a nucleic acid in such a way as to direct or regulate expression of the nucleic acid. Many methods for introducing nucleic acids into host cells, both in vivo and in vitro, are well known to those skilled in the art and include, without limitation, electroporation, calcium phosphate precipitation, polyethylene glycol (PEG) transformation, heat shock, lipofection, microinjection, and viral-mediated nucleic acid transfer.

Constructs as described herein can be introduced into a host cell. As used herein, “host cell” refers to the particular cell into which the nucleic acid is introduced and also includes the progeny or potential progeny of such a cell. A host cell can be any prokaryotic or eukaryotic cell. For example, nucleic acids can be expressed in bacterial cells such as E. coli, or in insect cells, yeast or mammalian cells (such as Chinese hamster ovary cells (CHO) or COS cells). Other suitable host cells are known to those skilled in the art.

As used herein, an “isolated” nucleic acid molecule is a nucleic acid molecule that is free of sequences that naturally flank one or both ends of the nucleic acid in the genome of the organism from which the isolated nucleic acid molecule is derived (e.g., a cDNA or genomic DNA fragment produced by PCR or restriction endonuclease digestion). Such an isolated nucleic acid molecule is generally introduced into a construct (e.g., a cloning vector, or an expression construct) for convenience of manipulation or to generate a fusion nucleic acid molecule. In addition, an isolated nucleic acid molecule can include an engineered nucleic acid molecule such as a recombinant or a synthetic nucleic acid molecule.

As used herein, a “purified” polypeptide is a polypeptide that has been separated or purified from cellular components that naturally accompany it. Typically, the polypeptide is considered “purified” when it is at least 70% (e.g., at least 75%, 80%, 85%, 90%, 95%, or 99%) by dry weight, free from the proteins and naturally occurring molecules with which it is naturally associated. Since a polypeptide that is chemically synthesized is, by nature, separated from the components that naturally accompany it, a synthetic polypeptide is “purified.”

Nucleic acids can be isolated using techniques known in the art. For example, nucleic acids can be isolated using any method including, without limitation, recombinant nucleic acid technology and/or the polymerase chain reaction (PCR). General PCR techniques are described, for example in PCR Primer: A Laboratory Manual, Dieffenbach & Dveksler, Eds., Cold Spring Harbor Laboratory Press, 1995. Recombinant nucleic acid techniques include, for example, restriction enzyme digestion and ligation, which can be used to isolate a nucleic acid. Isolated nucleic acids also can be chemically synthesized, either as a single nucleic acid molecule or as a series of oligonucleotides.

Polypeptides can be purified from natural sources (e.g., a biological sample) by known methods such as DEAE ion exchange, gel filtration, and hydroxyapatite chromatography. A polypeptide also can be purified, for example, by expressing a nucleic acid in an expression construct. In addition, a purified polypeptide can be obtained by chemical synthesis. The extent of purity of a polypeptide can be measured using any appropriate method, e.g., column chromatography, polyacrylamide gel electrophoresis, or HPLC analysis.

Nucleic acids can be detected using any number of amplification techniques (see, e.g., PCR Primer: A Laboratory Manual, 1995, Dieffenbach & Dveksler, Eds., Cold Spring Harbor Laboratory Press, Cold Spring Harbor, NY; and U.S. Pat. Nos. 4,683,195; 4,683,202; 4,800,159; and 4,965,188) with an appropriate pair of oligonucleotides (e.g., primers). A number of modifications to the original PCR have been developed and can be used to detect a nucleic acid.

Nucleic acids also can be detected using hybridization. Hybridization between nucleic acids is discussed in detail in Sambrook et al. (1989, Molecular Cloning: A Laboratory Manual, 2nd Ed., Cold Spring Harbor Laboratory Press, Cold Spring Harbor, NY; Sections 7.37-7.57, 9.47-9.57, 11.7-11.8, and 11.45-11.57). Sambrook et al. discloses suitable Southern blot conditions for oligonucleotide probes less than about 100 nucleotides (Sections 11.45-11.46). The Tm between a sequence that is less than 100 nucleotides in length and a second sequence can be calculated using the formula provided in Section 11.46. Sambrook et al. additionally discloses Southern blot conditions for oligonucleotide probes greater than about 100 nucleotides (see Sections 9.47-9.54). The Tm between a sequence greater than 100 nucleotides in length and a second sequence can be calculated using the formula provided in Sections 9.50-9.51 of Sambrook et al.

The conditions under which membranes containing nucleic acids are prehybridized and hybridized, as well as the conditions under which membranes containing nucleic acids are washed to remove excess and non-specifically bound probe, can play a significant role in the stringency of the hybridization. Such hybridizations and washes can be performed, where appropriate, under moderate or high stringency conditions. For example, washing conditions can be made more stringent by decreasing the salt concentration in the wash solutions and/or by increasing the temperature at which the washes are performed.

In addition, interpreting the amount of hybridization can be affected, for example, by the specific activity of the labeled oligonucleotide probe, by the number of probe-binding sites on the template nucleic acid to which the probe has hybridized, and by the amount of exposure of an autoradiograph or other detection medium. It will be readily appreciated by those of ordinary skill in the art that although any number of hybridization and washing conditions can be used to examine hybridization of a probe nucleic acid molecule to immobilized target nucleic acids, it is more important to examine hybridization of a probe to target nucleic acids under identical hybridization, washing, and exposure conditions. Preferably, the target nucleic acids are on the same membrane.

A nucleic acid molecule is deemed to hybridize to a nucleic acid but not to another nucleic acid if hybridization to a nucleic acid is at least 5-fold (e.g., at least 6-fold, 7-fold, 8-fold, 9-fold, 10-fold, 20-fold, 50-fold, or 100-fold) greater than hybridization to another nucleic acid. The amount of hybridization can be quantitated directly on a membrane or from an autoradiograph using, for example, a Phosphorlmager or a Densitometer (Molecular Dynamics, Sunnyvale, CA).

Polypeptides can be detected using antibodies. Techniques for detecting polypeptides using antibodies include enzyme linked immunosorbent assays (ELISAs), Western blots, immunoprecipitations and immunofluorescence. An antibody can be polyclonal or monoclonal. An antibody having specific binding affinity for a polypeptide can be generated using methods well known in the art. The antibody can be attached to a solid support such as a microtiter plate using methods known in the art. In the presence of a polypeptide, an antibody-polypeptide complex is formed.

Detection (e.g., of an amplification product, a hybridization complex, or a polypeptide) is usually accomplished using detectable labels. The term “label” is intended to encompass the use of direct labels as well as indirect labels. Detectable labels include enzymes, prosthetic groups, fluorescent materials, luminescent materials, bioluminescent materials, and radioactive materials.

A skilled artisan will appreciate that changes can be introduced into a nucleic acid molecule (e.g., into SEQ ID NOs: 1, 3, 5, 7, 9, 11, 13 or 15), thereby leading to changes in the amino acid sequence of the encoded polypeptide (e.g., SEQ ID NOs: 2, 4, 6, 8, 10, 12, 14 or 16). For example, changes can be introduced into nucleic acid coding sequences using mutagenesis (e.g., site-directed mutagenesis, PCR-mediated mutagenesis) or by chemically synthesizing a nucleic acid molecule having such changes. Such nucleic acid changes can lead to conservative and/or non-conservative amino acid substitutions at one or more amino acid residues. A “conservative amino acid substitution” is one in which one amino acid residue is replaced with a different amino acid residue having a similar side chain (see, for example, Dayhoff et al. (1978, in Atlas of Protein Sequence and Structure, 5(Suppl. 3):345-352), which provides frequency tables for amino acid substitutions), and a non-conservative substitution is one in which an amino acid residue is replaced with an amino acid residue that does not have a similar side chain.

A skilled artisan will appreciate that a nucleic acid molecule into which one or more changes have been introduced (e.g., SEQ ID NOs: 1, 3, 5, 7, 9, 11, 13 or 15), thereby leading to changes in the amino acid sequence of the encoded polypeptide (e.g., SEQ ID NOs: 2, 4, 6, 8, 10, 12, 14 or 16), can be used in the constructs and methods described herein. For example, nucleic acids and polypeptides that differ in sequence from SEQ ID NOs: 1, 3, 5, 7, 9, 11, 13 or 15 and SEQ ID NOs: 2, 4, 6, 8, 10, 12, 14 or 16, can have at least 80% sequence identity (e.g., at least 85%, 90%, 95%, or 99% sequence identity) to SEQ ID NOs: 1, 3, 5, 7, 9, 11, 13 or 15 and SEQ ID NOs: 2, 4, 6, 8, 10, 12, 14 or 16, respectively.

In calculating percent sequence identity, two sequences are aligned and the number of identical matches of nucleotides or amino acid residues between the two sequences is determined. The number of identical matches is divided by the length of the aligned region (i.e., the number of aligned nucleotides or amino acid residues) and multiplied by 100 to arrive at a percent sequence identity value. It will be appreciated that the length of the aligned region can be a portion of one or both sequences up to the full-length size of the shortest sequence. It also will be appreciated that a single sequence can align with more than one other sequence and hence, can have different percent sequence identity values over each aligned region.

The alignment of two or more sequences to determine percent sequence identity can be performed using the algorithm described by Altschul et al. (1997, Nucleic Acids Res., 25:3389 3402) as incorporated into BLAST (Basic Local Alignment Search Tool) programs, available at ncbi.nlm.nih.gov on the World Wide Web. BLASTN is the program used to align and compare the identity between nucleic acid sequences, while BLASTP is the program used to align and compare the identity between amino acid sequences. When utilizing BLAST programs to calculate the percent identity between a sequence and another sequence, the default parameters of the respective programs generally are used.

### Plants and Methods of Making

Transgenic plants are provided that contain a nucleic acid construct as described herein. Such transgenic plants exhibit an increase in the amount of TAGs in vegetative tissues relative to a plant lacking or not expressing the construct.

Plants that can be made transgenic using the compositions and methods described herein include, without limitation, sorghum, sugarcane, Miscanthus, maize, rye, and switch grass. As described herein, the presence of increased fatty acids in vegetative tissues was observed in two very different sorghum varieties, sweet sorghum and grain sorghum.

Sweet sorghum refers to high biomass sorghum varieties that accumulate sucrose or “sugar” in their stems or stalks. These varieties can be used for applications including ethanol, molasses, and forage production. The sweet sorghum variety, Ramada, was used as a vegetative oil production platform in the experiments described herein, but any number of sweet sorghum varieties could be similarly used.

Grain sorghum or milo refers to sorghum varieties that are used for production of grain for human and livestock consumption. These varieties typically have less biomass than sweet sorghum varieties. The grain sorghum variety, Texas430, was used as a vegetative oil production platform in the experiments described herein, but any number of grain sorghum varieties could be similarly used.

In addition, high oil grasses can be made transgenic using the compositions and methods described herein. High oil grasses can add energy density to forage for cattle feed, and can be used to make silage.

Methods of introducing a nucleic acid (e.g., a nucleic acid construct) into plant cells are known in the art and include, for example, particle bombardment, Agrobacterium-mediated transformation, microinjection, polyethylene glycol-mediated transformation (e.g., of protoplasts, see, for example, Yoo et al. (2007, Nature Protocols, 2(7):1565-72)), liposome-mediated DNA uptake, or electroporation. Following transformation, the transgenic plant cells can be regenerated into transgenic plants. As described herein, expression of the nucleic acid construct results in plants that exhibit an increase in the amount of TAGs in vegetative tissues relative to a plant not expressing the nucleic acid construct.

Following transformation, cells can be regenerated into T₀ transgenic plants or a subsequent generation of plants (e.g., T₁, T₂, T₃, etc.), which can be screened for expression of one or more genes from the nucleic acid construct and/or the amount of TAGs in the vegetative tissues. Screening for plants expressing one or more genes from the nucleic acid construct or for the amount of TAGs in the vegetative tissues can be performed using methods routine in the art (e.g., immunoassay, chromatography). Plants having increased amounts of TAGs in the vegetative tissues (compared to the amount of TAGs in the vegetative tissues of a corresponding plant lacking the construct) can be selected and used, for example, in a breeding program as discussed herein.

As used herein, an increase in the amount of TAGs in the vegetative tissues of the plant refers to an increase (e.g., a statistically significant increase) in the amount of TAGs in vegetative tissues by at least about 5% up to about 95% (e.g., about 5% to about 10%, about 5% to about 20%, about 5% to about 50%, about 5% to about 75%, about 10% to about 25%, about 10% to about 50%, about 10% to about 90%, about 20% to about 40%, about 20% to about 60%, about 20% to about 80%, about 25% to about 75%, about 50% to about 75%, about 50% to about 85%, about 50% to about 95%, and about 75% to about 95%) relative to the amount of TAGs in vegetative tissues from a corresponding plant lacking the nucleic acid construct grown under corresponding conditions. As used herein, statistical significance refers to a p-value of less than 0.05, e.g., a p-value of less than 0.025 or a p-value of less than 0.01, using an appropriate measure of statistical significance, e.g., a one-tailed two sample t-test.

A transgenic plant as described herein can be used in a plant breeding program to create new and useful cultivars, lines, varieties and hybrids. Thus, in some embodiments, a T₁, T₂, T₃ or later generation plant containing the nucleic acid construct can be crossed with a second plant, and progeny of the cross in which the construct is present can be identified. It will be appreciated that the second plant can exhibit a phenotypic trait such as, for example, disease resistance, high yield, leaf quality, height, plant maturation, stalk size, and/or leaf number per plant. In some instances, the second plant can express the same or a different transgene or nucleic acid construct as the plant to which it is crossed. Additionally or alternatively, the second plant can have one or more mutations, or be a wild-type plant.

Plant breeding is carried out using known procedures. DNA fingerprinting, SNP or similar technologies may be used in a marker-assisted selection (MAS) breeding program to transfer or breed mutant alleles into other lines, varieties or cultivars, as described herein. Progeny of the cross can be screened for the construct, expression of the construct, or the phenotype using methods described herein, and plants having the desired feature can be selected. For example, plants in the backcross generations (BC₁) can be screened using one or more of the methods described herein. Plants also can be screened for the amount of TAGs in the vegetative tissues, and those plants having the desired phenotype, compared to a corresponding plant that lacks the construct, can be selected. Plants identified as possessing the nucleic acid construct or the desired phenotype can be backcrossed or self-pollinated to create a second population to be screened. Backcrossing or other breeding procedures can be repeated until the desired phenotype of the recurrent parent is recovered.

Successful crosses yield F₁ plants that are fertile and that can be backcrossed with one of the parents if desired. In some embodiments, a plant population in the F₂ generation is screened for the appropriate gene expression using standard methods (e.g., PCR). Selected plants then can be crossed with one of the parents and the first backcross (BC₁) generation plants can be self-pollinated to produce a BC₁F₂ population that is again screened for appropriate gene expression. The process of backcrossing, self-pollination, and screening can be repeated, for example, four or more times until the final screening produces a plant that is fertile and reasonably similar to the recurrent parent. This plant, if desired, can be self-pollinated and the progeny can be subsequently screened again to confirm that the plant expresses the appropriate sequences and exhibits the proper phenotype. Breeder's seed of the selected plant can be produced using standard methods including, for example, field testing, genetic analysis, and/or confirmation of the phenotype.

The result of a plant breeding program using the transgenic plants described herein are new and useful cultivars, varieties, lines, and hybrids. As used herein, the term “variety” refers to a population of plants that share constant characteristics which separate them from other plants of the same species. A variety is often, although not always, sold commercially. While possessing one or more distinctive traits, a variety can be further characterized by a very small overall variation between individuals with that variety. A “pure line” variety may be created by several generations of self-pollination and selection, or vegetative propagation from a single parent using tissue or cell culture techniques. A “line,” as distinguished from a variety, most often denotes a group of plants used non-commercially, for example, in plant research. A line typically displays little overall variation between individuals for one or more traits of interest, although there may be some variation between individuals for other traits.

Depending on the plant, hybrids can be produced by preventing self-pollination of female parent plants (i.e., seed parents) of a first variety, permitting pollen from male parent plants of a second variety to fertilize the female parent plants, and allowing F₁ hybrid seeds to form on the female plants. Self-pollination of female plants can be prevented by emasculating the flowers at an early stage of flower development. Alternatively, pollen formation can be prevented on the female parent plants using a form of male sterility. For example, male sterility can be produced by cytoplasmic male sterility (CMS), nuclear male sterility, genetic male sterility, molecular male sterility wherein a transgene inhibits microsporogenesis and/or pollen formation, or self-incompatibility. Female parent plants containing CMS are particularly useful. In embodiments in which the female parent plants are CMS, the male parent plants typically contain a fertility restorer gene to ensure that the F₁ hybrids are fertile. In other embodiments in which the female parents are CMS, male parents can be used that do not contain a fertility restorer. F₁ hybrids produced from such parents are male sterile. Male sterile hybrid seed can be interplanted with male fertile seed to provide pollen for seed-set on the resulting male sterile plants.

In accordance with the present invention, there may be employed molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### EXAMPLES

### Example 1—Plant Material, Growth, and Sampling Conditions

Sorghum (Sorghum bicolor L. Moench. TX430 genotype) seeds were sown into jiffy pots with soil mix composed of Metromix 300 (Sun Gro Horticulture, Agawam, MA) and grown under the greenhouse condition utilizing a 12/12 h photoperiod with day/night temperatures of 27˜29° C./19˜21° C. For field trials, transgenic sorghum event and wild-type plants were grown in the field at Eastern Nebraska Research and Education Center (ENREC) in Mead, NE, USA (41° 08′42.7″N 96° 26′20.5″W) in 2021 and 2022 seasons. To measure lipid content in vegetative tissues at fields, leaves and stalks were harvested from 5′ node count from the bottom. Tissues were collected in three different stages; V5 (fully expanded leaves but before flag leaf appears), anthesis (flowering), and seed-filling (soft-dough) stages. Samples were immediately frozen with liquid nitrogen and then lyophilized by freeze-dryer (Labconco FreeZone 2.5). After the lyophilization, samples were immediately analyzed or stored in a −80° C. freezer until analysis.

### Example 2—Binary Vectors

Binary vectors for sorghum plants were constructed by GoldenBraid modular assembly (Sarrion-Perdigones et al., 2013, Plant Phys., 162:1618-31). Genes used in vector construction were GoldenBraid-domesticated and codon-optimized for sorghum by gene synthesis (GenScript Biotech, Piscataway, NJ). Genes used in this study is listed; Arabidopsis DGAT1 (AtDGAT1, NP_179535.1), Cuphea avigera var. pulcherrima DGAT1 (CpuDGAT1, ANN46862.1), sorghum Wrinkled1 (SbWri1, XP_002450194.1), sesame oleosin (SiOle, Q9XHP2.1), Cuphea viscosissima FatB1 (CvFatB1, G3ESU9.1), Cuphea viscosissima LPAT (CvLPAT, ALM22867.1), Cuphea palustris thioesterase (Thio14, AAC49180.1), and Cuphea avigera var. pulcherrima (CpuLPATB, ALM22873.1). For sorghum Wrinkled1, one amino acid residue was mutated (K10R, here in mSbWri1) to increase protein stability (Zhai et al., 2017, The Plant Cell, 29:871-89). Each gene was driven by constitutive promoters such as maize ubiquitin1, sugarcane ubiquitin4, and rice PGD1.

### Example 3—Sorghum Transformation

Agrobacterium tumefaciens cells (strain NTL4/Chry5) containing sorghum oil binary vectors were obtained by electroporation. Sorghum plants were transformed using immature embryos as described previously (Howe et al., 2006, Plant Cell Reports, 25:784-91). One day prior to inoculation, Agrobacterium was shaking-incubated in 50 ml of liquid LB with the appropriate antibiotics and grown for 8 h at 28° C. The bacteria were harvested by centrifugation and the pellet was resuspended in AB minimal medium to a final OD₆₅₀=0.2 and incubated overnight with agitation at 28° C. The bacteria were harvested and resuspended in co-culture medium added with 200 μM acetosyringone to a final OD₆₅₀=0.4. Immature embryos were isolated from fresh sorghum heads. Before isolation, heads were sterilized by submerging in 50% Ultra Clorox with 2 drops Tween 20 under the agitation at room temperature for 20 min. sterilized heads were rinsed with distilled water three times and then allowed to air dry in a laminar flow hood. Individual immature seeds were isolated from the heads and processed additional sterilization steps in 70% ethanol for 1 min followed by rinse with distilled water one time and further submerging in 30% Ultra Clorox for 20 min. After rinse with distilled water three times, seeds were air dried in a laminar flow hood briefly. Immature embryos were isolated from seeds into sterile petri plates containing 1 ml co-culture media supplemented with 300 mM acetosyringone. Co-culture media was replaced to 1 ml of Agrobacterium inoculum and incubated further 30 min then remove the liquid. Co-cultured embryos were transferred scutellum side up into new petri plates containing 4 sterile Whatman filter papers saturated with 4 ml of co-culture with 300 mM of acetosyringone. Wrapped co-culture plates were placed at 24° C. dark for 2 days. After co-cultivation, embryos were transferred to Elkonin's selection plate with 20 mg/L G418 and further grown at 28° C. in the dark for 2˜3 weeks. transfer the embryos with developed callus were transferred to R1 regeneration media with 10 mg/L G418 and placed in the 24° C. incubator with 16/8 hour photoperiod. One month later, plantlets with roots and shoots were transferred into soil and grown in the 28° C. incubator with a 16/8 hour photoperiod.

### Example 4 DNA Extraction and Genotyping PCR

Genomic DNA was extracted from sorghum leaves using cetyl trimethylammonium bromide (CTAB) method. About 20 ng of DNA was used for PCR using Promega Go Taq™ Master Mix in a volume of 20 μl with gene-specific primer sets (Table 1). The PCR reaction was programmed as using a touchdown protocol: 95° C. for 2 minutes, and 35 cycles of 95° C. for 15 sec, 65° C. (−0.3° C./cycle) for 30 sec, 72° C. for 1 min. the PCR was terminated at 72° C. for 5 minutes and cooling down to 4° C.

### Example 5—DNA-Gel Blot Analysis

Total genomic DNA was digested with SmaI restriction enzyme (ThermoScientific) and separated on 0.7% agarose gels. The gel was treated sequentially with depurination, denaturation and neutralization solutions and then transferred to Hybond-XL nylon membrane (GE Healthcare Life Sciences, Chicago, IL) by capillary transfer method. The blots were hybridized with ³²P-labeled CpuDGAT1 probes under high-stringency conditions (65° C.) and visualized using Typhoon FLA 7000 phosphorimaging system (GE Healthcare Life Sciences).

Example 6—RNA Isolation and Droplet Digital PCR Total RNA was isolated from mature leaves of wild-type and transgenic sorghum plants using a RNeasy Plant Mini Kit according to the manufacturer's protocol (Qiagen). RNA was clean from DNA contamination using TURBO DNA-free™ Kit (ThermoScientific). First strand cDNA was synthesized from 1 μg of total RNA with High-Capacity cDNA Reverse Transcription Kit (ThermoScientific). Droplet digital PCR (ddPCR) was performed using gene-specific primer sets (Table 1) in a total volume of 20 ml, using 5 μl of 25× diluted cDNA was sued for ddPCR according to the manufacturer's protocol (BioRad). The data were normalized to a sorghum reference gene Eukaryotic initiation factor-4A, EIF4A (Sudhakar Reddy et al., 2016, Front. Plant Sci., 7:529).

### Example 7—Extraction of TAG and Analysis of Sorghum Vegetative Tissues

Total lipid was extracted from vegetative tissues using a modification of the Bligh-Dyer method (Bligh and Dyer, 1959, Canadian J. Biochem. Biophysiol., 37:911-7). A 30 mg aliquot of freeze-dried sorghum leaves was ground in 3 ml of methanol:chloroform (2:1 v/v) with 20 mg of tri 17:0-TAG (Nu-Chek Prep, Elysian, MN, USA) as an internal standard. Homogenized samples were shake-incubated for 30 min at room temperature, and lipids were partitioned and extracted by adding 1 ml of chloroform and 1.9 ml of distilled water. Extracted organic phase was fully evaporated using nitrogen gas and resuspended in 100 ml of chloroform. To separate TAG, TLC was developed using the 50 μl of lipid extract using mobile phase (heptane:diethyl ether:acetic acid, 70:30:1, v/v/v). TAG was visualized and scraped by spraying of 0.01% primuline (dissolved in 80% acetone, w/v; Sigma-Aldrich, St. Louis, MO) on TLC plate. Fatty acid methyl esters (FAMEs) were generated by adding 1 ml of 2.5% H2SO4 (v/v) in methanol on both total lipids and TAG samples and heating for 30 min at 90° C. Partitioned FAMEs were analyzed by Agilent Technologies 7890A GC with a 30 m×0.25 mm HP-INNOWax column (Agilent, Santa Clara, CA). The gas chromatograph was programmed for an initial temperature of 90° C. (1 min hold) followed by an increase of 30° C./min⁻¹ to 235° C. and maintained for a further 5 min. Detection was achieved using flame ionization (FID). For sorghum stalk lipid analysis, a 60 mg aliquot of freeze-dried samples was used.

### Example 8—Acyl-ACP Thioesterase Activity Assay

Total crude protein extracts were prepared by grinding sorghum leaves in 100 mM Tris (pH 8.5) buffer with 1 mM EDTA. After centrifugation to remove cell debris, enzyme activity assay was carried out similar to the method described previously (Gan et al., 2022, PNAS USA, 119:e2201160119). 50 ml of reaction volume consists of 50 mg of soluble protein extract with 1000 DPM [1-¹⁴C] acyl-ACP (myristoyl-ACP, 14:0 or oleoyl-ACP, 18:1A9; 55 mCi/mmol) in 100 mM Tris (pH 8.5) buffer at room temperature for 5 min. Both acyl-ACPs were enzymatically synthesized using recombinant spinach ACP (Rock and Garwin, 1979, J. Biol. Chem., 254:7123-8). After 5 min, the reaction was terminated by adding 50 ml of 1 M acetic acid in isopropanol. Free FAs produced by thioesterase were extracted with heptane saturated with 50% isopropanol (v/v) three times. Residual acyl-ACPs were collected from a lower phase of reactant separately. Free FAs and unreacted acyl-ACPs were dried under nitrogen gas and re-dissolved in 15 mL of scintillation mixture (Bio-Safe II, Research Products International). Radioactivity was measured using liquid scintillation counter (Beckman Coulter LS6500).

TABLE 1
Primers used in genotyping PCR and droplet digital PCR

| Primer name | Sequence | SEQ ID NO |
|---|---|---|
| EIF4α ddPCR F | 5′-caactttgtcacccgcgatga-3′ | 17 |
| EIF4α ddPCR R | 5′-tccagaaaccttagcagccca-3′ | 18 |
| SbWri1 genotyping F | 5′-aagccgctgctagggcttac-3′ | 19 |
| SbWri1 genotyping R | 5′-gattgatgctggagccgaag-3′ | 20 |
| SbWri1 ddPCR F | 5′-gcgaggctaagacaccagac-3′ | 21 |
| SbWri1 ddPCR R | 5′-gccgatattgctgtcgaaat-3′ | 22 |
| AtDGAT1 genotyping ddPCR F | 5′-ggcagacaaggcaaatccag-3′ | 23 |
| AtDGAT1 genotyping R | 5′-cctacaagggactgcgatgc-3′ | 24 |
| AtDGAT1 ddPCR R | 5′-tgggttgatgtactgctcgat-3′ | 25 |
| SiOle genotyping ddPCR F | 5′-cccacctccaactccaacct-3′ | 26 |
| SiOle genotyping R | 5′-ggtctggctacctgcgactg-3′ | 27 |
| SiOle ddPCR R | 5′-ccaggaggaaaatcgtgatg-3′ | 28 |
| CpuDGAT1 genotyping F | 5′-atcctggtgaacgtggtcat-3′ | 29 |
| CpuDGAT1 genotyping R | 5′-gagctcagccacaatgttca-3′ | 30 |
| CvFatB1 genotyping ddPCR F | 5′-aacacctggttcagccagtc-3′ | 31 |
| CvFatB1 genotyping ddPCR R | 5′-gtgtgggctatccacgaagt-3′ | 32 |
| CvLPAT2 genotyping ddPCR F | 5′-ctgggcaatcctgtgaagat-3′ | 33 |
| CvLPAT2 genotyping ddPCR R | 5′-agccagagcgtagagctgtc-3′ | 34 |
| Thio14 genotyping ddPCR F | 5′-tcggaaggactcctgaaatg-3′ | 35 |
| Thio14 genotyping ddPCR R | 5′-caaacagacgtggccctaat-3′ | 36 |
| CpuLPATB genotyping ddPCR F | 5′-ctgggcaatcctgtgaagat-3′ | 37 |
| CpuLPATB genotyping ddPCR R | 5′-agccagagcgtagagctgtc-3′ | 38 |

### Example 9—Nucleotide and Polypeptide Sequences of the Genes

The following sequences were used as described herein.

>CpuDGAT1
(SEQ ID NO: 1)
atgcacgaggccgtgagccacttcctgcacaggcacgctccactcagcctgtccggcttcgc
catggctatcgtgtccggcaccctcggagtggctgcttccagcttcatccctgacagcgatc
actccaccacaagcccctccctgaggaagcgcaactccagctccctcttcccgaaggccagc
gacaccagctccgtggatggcaaggccgctcacaggacaagctccccagtccacctcaagct
ggctgagtccccgctgagctcccgcaacatcttcaagcagaatcacgagggcctcttcaacc
tgtgcatggtcacactcgtggccgtcatcatcaggctcttcctggagaatctcctgaagtac
ggctggctgatgaagcgcgacttctggctcagcaccttcacagcctggcctctgttcatctg
ctccctgggcctgccaatcttccctctggctgctttcgtggtcgagaagctcgctcagaaga
atctcctgcctgagcccatcgtgctgtgcagccacgtcatcatcacatccgcttccgtgctc
taccctgctctcgtcatcctgaggttcgactgcgccctgatgagcggcatcggcctcatgct
ctactcctgcgccctctggctgaagctcgtgagctacgctcacacatcctacgatatgcgct
gcgaggctaagagccgcctcgagggcaagagctccgctgactccaagaacggcgagctgcct
tacagggtgaatatcaaggatctcgcctacttcatggtcgctcctaccctctgctaccagct
gagctaccccaggacacagttcatccgcaagttctgggtggcccgccaggtcctgaagctca
tcctggtgaacgtggtcatgggcttcatcatcgagcagtacatgatccctgtcatgcacaac
tccaagccaccgaggcgcggatactggctgcacttcatcgagcgcaatctcaagctggctgt
gcccagcatcggcctctggttctgcatcttctactccatcttccacctctggctgaacattg
tggctgagctcctgaggttcggcgaccgcgagttctacaaggattggtggaacgctaagaat
atggaggagtactggaagatgtggaatatcccagtgcacaggtggatggtccgccacctgta
cggcccatgcatgaagaggaagctcccgcgctgggtggccatcagcatctccttcctcctga
gcgctgtcctccacgagatctgcgtgtccgtcccatgccacgtgttccagctgtgggccttc
aacggcatgatgctccagatcccgctcgtcctgagctccaagcctctgcagaagaggttccc
cagctccaaggccggcaacgtgttcttctggttcctcttctgcatctacggccagccgaatt
gcgtcctcatgtactaccatgctctgatggagaggcgcggcctccgcatcgattga
(SEQ ID NO: 2)
MHEAVSHFLHRHAPLSLSGFAMAIVSGTLGVAASSFIPDSDHSTTSPSLRKRNSSSLFPKAS
DTSSVDGKAAHRTSSPVHLKLAESPLSSRNIFKQNHEGLFNLCMVTLVAVIIRLFLENLLKY
GWLMKRDFWLSTFTAWPLFICSLGLPIFPLAAFVVEKLAQKNLLPEPIVLCSHVIITSASVL
YPALVILRFDCALMSGIGLMLYSCALWLKLVSYAHTSYDMRCEAKSRLEGKSSADSKNGELP
YRVNIKDLAYFMVAPTLCYQLSYPRTQFIRKFWVARQVLKLILVNVVMGFIIEQYMIPVMHN
SKPPRRGYWLHFIERNLKLAVPSIGLWFCIFYSIFHLWLNIVAELLRFGDREFYKDWWNAKN
MEEYWKMWNIPVHRWMVRHLYGPCMKRKLPRWVAISISFLLSAVLHEICVSVPCHVFQLWAF
NGMMLQIPLVLSSKPLQKRFPSSKAGNVFFWFLFCIYGQPNCVLMYYHALMERRGLRID
>mSbWri1
(SEQ ID NO: 3)
atggatatggagaggagccagcagcagcgctcccctacagagagcccaccgcctccctcccc
ctccagctccagctccagcgtgagcgctgacacagtcctcccaccgcctggcaagaggcgca
gggctgctaccacagctaaggccaaggctggcgccaagcctaagcgggctaggaaggatgct
gctgctgctgctgacccaccaccaccaccagcggctgctgctgctggcaagaggtccagcgt
gtaccgcggcgtcactaggcacaggtggacaggaaggttcgaggctcacctctgggataagc
actgcctcgccgctctgcacaacaagaagaagggcaggcaggtgtacctgggcgcctacgat
agcgaggaagccgctgctagggcttacgacctcgctgctctgaagtactggggcccagagac
actcctgaacttcccggtggaggactactccagcgagatgcccgagatggagggcgtgtccc
gggaggagtacctcgccagcctgcgcaggcgctccagcggcttctcccgcggcgtggctaag
tacaggggcgtcgctaggcaccaccacaatggccgctgggaggctaggattggaagggtctt
cggcaacaagtacctctacctgggcaccttcgatacacaggaagaggccgctaaggcctatg
acctcgctgctatcgagtaccgcggcgtgaacgctgtcaccaatttcgatatcagctgctac
ctcgaccacccactcttcctggcccagctccagcaggagcctcaggtggtcccagctctgaa
tcaagaggcccagccggaccagtccgagacagagacaatcgcccaggagagcgtgtccagcg
aggctaagacaccagacgataacgctgagccggacgataatgccgagcctgacgatatcgct
gagcccctgatcaccgtcgacgattccatcgaggagtccctctggagcccgtgcatggatta
cgagctggacacaatgtccaggagcaacttcggctccagcatcaatctctccgagtggttca
acgacgccgatttcgacagcaatatcggctgcctgttcgatggatgctccgctgtggatgag
ggcggcaaggatggagtcggcctcgctgatttctccctcctggaggacttctccctgttcga
ggctggcgacggccagctcaaggatgtgctgagcgacatggaggaaggcatccagccaccga
ccatgatctccgtctgcaactga
(SEQ ID NO: 4)
MDMERSQQQRSPTESPPPPSPSSSSSSVSADTVLPPPGKRRRAATTAKAKAGAKPKRARKDA
AAAADPPPPPAAAAAGKRSSVYRGVTRHRWTGRFEAHLWDKHCLAALHNKKKGRQVYLGAYD
SEEAAARAYDLAALKYWGPETLLNFPVEDYSSEMPEMEGVSREEYLASLRRRSSGFSRGVAK
YRGVARHHHNGRWEARIGRVFGNKYLYLGTFDTQEEAAKAYDLAAIEYRGVNAVTNFDISCY
LDHPLFLAQLQQEPQVVPALNQEAQPDQSETETIAQESVSSEAKTPDDNAEPDDNAEPDDIA
EPLITVDDSIEESLWSPCMDYELDTMSRSNFGSSINLSEWENDADFDSNIGCLEDGCSAVDE
GGKDGVGLADFSLLEDFSLFEAGDGQLKDVLSDMEEGIQPPTMISVCN
>Siole
(SEQ ID NO: 5)
Atggcgtgtcattatggtcaacaacaacaaacctgtgctccccacctccaactccaacctcg
ggcgtgtagggtcgtgaaggcagccaccgcagtcacagcaggaggctccctcctcgtgctct
caggtcttaccttggccggaacagttatcgcgctgacgattgcaactccactcctcgtgatc
ttctcccctgtgcttgtccccgctgtcatcacgattttcctcctgggcgccgggtttctcgc
ttcaggtggattcggcgttgccgctctctccgtgctgagctggatctacaggtatctgacgg
gcaagcacccaccgggggcagactgccttgagtcggcaaagactaagttggcctcctgtgct
cgcgagatgaaggatagagcggaacagttttcttgtcaaccagtcgcaggtagccagaccag
ctag
(SEQ ID NO: 6)
MACHYGQQQQTCAPHLQLQPRACRVVKAATAVTAGGSLLVLSGLTLAGTVIALTIATPLLVI
FSPVLVPAVITIFLLGAGFLASGGFGVAALSVLSWIYRYLTGKHPPGADCLESAKTKLASCA
REMKDRAEQFSCQPVAGSQTS
>CvFatB1
(SEQ ID NO: 7)
atggtggccgctgccgctacatccgctttcttccctgtcccagcgcctggcacaagccctaa
gcctggcaagtccggaaactggccttccagcctcagcccaaccttcaagccgaagtccatcc
ctaatggcggcttccaggtcaaggccaacgctagcgctcaccctaaggctaacggctccgct
gtgaatctcaagtccggctccctgaatacccaagaggacacatccagctccccaccgcctcg
cgctttcctcaaccagctgcccgattggagcatgctcctgaccgccatcaccacagtgttcg
tcgccgctgagaagcagtggacaatgctcgacaggaagtccaagcgcccagacatgctcgtg
gatagcgtcggcctgaagtccattgtgagggacggcctggtcagcaggcacagcttctccat
caggtcctacgagatcggcgctgataggaccgccagcatcgagacactcatgaatcacctgc
aggagacaacaatcaaccactgcaagtccctcggcctgcataatgatggattcggaaggacc
cctggaatgtgcaagaacgatctcatctgggtgctgacaaagatgcagatcatggtcaatag
gtaccctacctggggcgacacagtcgagatcaacacctggttcagccagtccggcaagatcg
gcatggccagcgactggctcatctccgattgcaataccggcgagatcctgatccgcgctaca
agcgtctgggccatgatgaaccagaagaccaggcgcttctcccgcctcccatacgaggtgag
gcaggagctgacaccccacttcgtggatagcccacacgtcatcgaggacaatgatcagaagc
tgaggaagttcgacgtcaagaccggcgattccatccgcaagggcctcacacccaggtggaac
gacctggatgtgaatcagcacgtgagcaacgtcaagtacatcggctggatcctcgagtccat
gccaatcgaggtgctggagacacaggagctctgctccctgacagtcgagtacaggcgcgagt
gcggcatggacagcgtgctcgagtccgtgacagctgtggatccttccgagaacggcggccgc
tcccagtacaagcacctcctgaggctggaggacggcaccgatatcgtcaagagcaggacaga
gtggcgccctaagaacgctggcaccaatggcgccatcagcacctccacagccaagacatcca
acggcaatagcgtgtcctga
(SEQ ID NO: 8)
MVAAAATSAFFPVPAPGTSPKPGKSGNWPSSLSPTFKPKSIPNGGFQVKANASAHPKANGSA
VNLKSGSLNTQEDTSSSPPPRAFLNQLPDWSMLLTAITTVFVAAEKQWTMLDRKSKRPDMLV
DSVGLKSIVRDGLVSRHSFSIRSYEIGADRTASIETLMNHLQETTINHCKSLGLHNDGFGRT
PGMCKNDLIWVLTKMQIMVNRYPTWGDTVEINTWFSQSGKIGMASDWLISDCNTGEILIRAT
SVWAMMNQKTRRFSRLPYEVRQELTPHFVDSPHVIEDNDQKLRKFDVKTGDSIRKGLTPRWN
DLDVNQHVSNVKYIGWILESMPIEVLETQELCSLTVEYRRECGMDSVLESVTAVDPSENGGR
SQYKHLLRLEDGTDIVKSRTEWRPKNAGTNGAISTSTAKTSNGNSVS
>CvLPAT2
(SEQ ID NO: 9)
atggccatcgccgctgccgctgtcatcttcctgttcggcctcatcttcttcgcctccggcct
gatcatcaacctcttccaggctctgtgcttcgtcctcatcaggccgctgtccaagaacgcct
acaggcgcatcaatcgcgtgttcgctgagctcctgctcagcgagctgctgtgcctcttcgac
tggtgggctggagctaagctgaagctcttcaccgatcctgagacattcaggctcatgggcaa
ggagcacgccctcgtgatcatcaaccacatgaccgagctcgactggatggtcggctgggtca
tgggccagcacttcggctgcctgggctccatcatcagcgtcgccaagaagtccacaaagttc
ctgccggtgctcggctggtccatgtggttcagcgagtacctgtacctcgagaggagctgggc
taaggacaagtccaccctcaagagccacatcgagcgcctgatcgattaccccctcccattct
ggctggtcatcttcgtggagggcacaaggttcacccgcacaaagctgctggctgctcagcag
tacgccgtgtcctccggcctccctgtccctaggaatgtgctgatcccccgcaccaagggctt
cgtgtcctgcgtgagccacatgcgctccttcgtccccgccgtgtacgacgtcacagtggctt
tcccaaagacctccccaccgcctacactgctcaatctcttcgagggccagagcatcatgctg
cacgtccacatcaagaggcacgccatgaaggacctgccagagagcgatgatgctgtggctga
gtggtgcagggataagttcgtggagaaggacgccctgctcgataagcacaacgctgaggata
ccttctccggccaagaggtgaggcacacaggctcccgccagctcaagagcctgctcgtggtc
atcagctgggtggtcgtgaccacattcggcgctctgaagttcctccagtggtccagctggaa
gggcaaggccttctccgctatcggcctgggcatcgtcaccctgctcatgcacgtgctgatcc
tctccagccaggccgagaggagcaaccctgctgaggtcgcccaggctaagctcaagaccggc
ctgtccatcagcaagaaggtgacagataaggagaattga
(SEQ ID NO: 10)
MAIAAAAVIFLFGLIFFASGLIINLFQALCFVLIRPLSKNAYRRINRVFAELLLSELLCLED
WWAGAKLKLFTDPETFRLMGKEHALVIINHMTELDWMVGWVMGQHFGCLGSIISVAKKSTKE
LPVLGWSMWFSEYLYLERSWAKDKSTLKSHIERLIDYPLPFWLVIFVEGTRFTRTKLLAAQQ
YAVSSGLPVPRNVLIPRTKGFVSCVSHMRSFVPAVYDVTVAFPKTSPPPTLLNLFEGQSIML
HVHIKRHAMKDLPESDDAVAEWCRDKFVEKDALLDKHNAEDTFSGQEVRHTGSRQLKSLLVV
ISWVVVTTFGALKFLQWSSWKGKAFSAIGLGIVTLLMHVLILSSQAERSNPAEVAQAKLKTG
LSISKKVIDKEN
>AtDGAT1
(SEQ ID NO: 11)
atggcgattctggattcggctggggtgactactgtgacggaaaacggggggggcgagttcgt
ggacctggataggctgcggcggcggaagagcaggtctgattccagcaacggactcctgcttt
caggctcggacaacaattccccaagcgacgatgtgggagcaccagctgacgtccgggataga
atcgacagcgtggtcaacgacgatgcccaagggacggccaatctggctggcgacaacaatgg
cggaggtgacaacaacggcggaggaaggggtggaggagagggaaggggtaacgcagatgcaa
ccttcacatacaggccatccgtgccagcacacaggagagcacgcgaaagccctctctcttca
gacgccatctttaagcagtcgcatgctggattgttcaatctctgcgttgtggtcttgattgc
cgtcaactctcggttgatcattgagaatctcatgaagtacggctggcttatcagaacggatt
tctggttttcgtccaggtccctgcgcgactggcctcttttcatgtgctgtatctctttgtca
attttccccctcgccgcttttactgtcgagaagttggttctccaaaagtatatcagcgaacc
agttgtgatttttctgcacatcattatcacgatgactgaggtcttgtacccggtctatgtta
ccctccggtgcgattcagcgttcctgtcgggcgttacccttatgttgctcacatgtatcgtt
tggctgaagcttgtgtcttacgcacatacatcatatgatattaggtcgctcgcgaacgcggc
agacaaggcaaatccagaggtgtcctactatgtcagcctgaagtctcttgcctacttcatgg
tggctccgaccctgtgctaccagccttcctatccccggagcgcctgtatcagaaaggggtgg
gttgccaggcaattcgctaagctcgtgatttttacaggattcatgggctttattatcgagca
gtacatcaacccaattgtcaggaatagcaagcacccgctgaagggcgacctcctttatgcta
tcgaacgcgtcttgaagctctccgttcctaacctctacgtgtggctttgcatgttctactgt
ttctttcatctgtggcttaacatcctcgccgagttgctctgcttcggagatcgggaatttta
caaggactggtggaacgctaagtccgtgggcgactattggagaatgtggaatatgcccgtcc
acaagtggatggttcggcatatctacttcccttgcctgagatctaagattcccaagaccctg
gcgattatcattgcatttcttgtgtcagcggtcttccacgagctgtgcatcgcagtcccttg
taggttgttcaagctctgggcgtttcttggcatcatgttccaggttcccttggtgtttatta
cgaactacctccaagaacgcttcgggtccactgtgggtaatatgatcttctggtttatcttc
tgcattttcgggcagccgatgtgtgtcctgctttactatcatgacctcatgaaccgcaaggg
ttcgatgtcctga
(SEQ ID NO: 12)
MAILDSAGVTTVTENGGGEFVDLDRLRRRKSRSDSSNGLLLSGSDNNSPSDDVGAPADVRDR
IDSVVNDDAQGTANLAGDNNGGGDNNGGGRGGGEGRGNADATFTYRPSVPAHRRARESPLSS
DAIFKQSHAGLFNLCVVVLIAVNSRLIIENLMKYGWLIRTDFWFSSRSLRDWPLEMCCISLS
IFPLAAFTVEKLVLQKYISEPVVIFLHIIITMTEVLYPVYVTLRCDSAFLSGVTLMLLTCIV
WLKLVSYAHTSYDIRSLANAADKANPEVSYYVSLKSLAYFMVAPTLCYQPSYPRSACIRKGW
VARQFAKLVIFTGEMGFIIEQYINPIVRNSKHPLKGDLLYAIERVLKLSVPNLYVWLCMFYC
FFHLWLNILAELLCFGDREFYKDWWNAKSVGDYWRMWNMPVHKWMVRHIYFPCLRSKIPKTL
AIIIAFLVSAVFHELCIAVPCRLFKLWAFLGIMFQVPLVFITNYLQERFGSTVGNMIFWFIF
CIFGQPMCVLLYYHDLMNRKGSMS
>Thio14
(SEQ ID NO: 13)
atggttgcagctgctgcaagtgccgcattcttttctgtagctacaccgagaactaatatctc
tccatcatccctcagtgttccgtttaagccaaagagtaatcataacggcgggttccaagtta
aggcgaacgcttcagctcatccgaaggcaaacgggagtgcagtttccctcaaatcaggctct
cttgagactcaggaggacaagacaagctcaagtagtccacctcctcgaactttcattaatca
gcttcctgtttggtcaatgttactttcagctgtcacaactgtttttggcgtggctgagaagc
agtggcctatgttggatcgtaaaagtaagaggccagacatgttagttgagcctctgggagtt
gatagaattgtttatgatggagtttcattccgtcaatcgttcagcattcgatcctacgaaat
aggagccgacagaactgcttcaattgaaacccttatgaatatgtttcaagaaacttcactaa
accactgtaaaattataggacttcttaatgatggcttcggaaggactcctgaaatgtgtaag
agagatttgatatgggttgttactaagatgcaaatcgaggtgaacagatatccgacttgggg
agatactattgaggttaacacttgggtttctgcaagcggcaaacatggtatgggacgagatt
ggcttatctcagattgtcacacaggtgagatattgattagggccacgtctgtttgggcaatg
atgaaccaaaagactagaaggctatcaaagataccttacgaagttaggcaagaaatagaacc
gcagtttgtagattcagcgccagttatcgttgatgacagaaagtttcataaacttgatctta
aaactggagattccatttgtaacggtcttactccgaggtggacagatttggatgtgaaccag
cacgtaaacaacgttaaatatatcgggtggatactccagtctgttcctactgaggttttcga
gactcaggagttatgtggactcacgcttgagtacagaagagagtgcggacgtgactccgttc
tggagagcgttactgctatggacccttcaaaagaaggggatcgttccttgtaccaacatctt
cttcgtttagaagatggagcagatatcgtgaaagggagaaccgagtggcgtccaaaaaatgc
gggagcaaagggagccattcttacaggaaagacaagcaacggaaattcaatctcttga
(SEQ ID NO: 14)
MVAAAASAAFFSVATPRINISPSSLSVPFKPKSNHNGGFQVKANASAHPKANGSAVSLKSGS
LETQEDKTSSSSPPPRTFINQLPVWSMLLSAVTTVFGVAEKQWPMLDRKSKRPDMLVEPLGV
DRIVYDGVSFRQSFSIRSYEIGADRTASIETLMNMFQETSLNHCKIIGLLNDGFGRTPEMCK
RDLIWVVTKMQIEVNRYPTWGDTIEVNTWVSASGKHGMGRDWLISDCHTGEILIRATSVWAM
MNQKTRRLSKIPYEVRQEIEPQFVDSAPVIVDDRKFHKLDLKTGDSICNGLTPRWTDLDVNQ
HVNNVKYIGWILQSVPTEVFETQELCGLTLEYRRECGRDSVLESVTAMDPSKEGDRSLYQHL
LRLEDGADIVKGRTEWRPKNAGAKGAILTGKTSNGNSIS
>CpuLPATB
(SEQ ID NO: 15)
Atggcctccatcggcatctccagcctcctgaagaacaggaagctggagtccagcttcagcac
aggcttcgctaaggattccttccctcacagccccgagaagacaatgatcagggacgattccg
agcacaggaccatcatcgctgatggcctcagcgtggacgatgacgatggctggatggccgtg
ctgatctcctgggctcgcctcgtcatgtgcttcgtgctcgtcctgatcaccacaagcatctg
gaccctgatcatggtcatcctcatcccttggccctgcgagaggatcaagcagtccaacgtgt
tcggccacgtcagcggccgcatgctgatgtggctcctgggcaatcctgtgaagattgaggga
gctgagcatgctaacgagagggccatctacatctgcaatcacgcttccccactcgatatcgt
cctgaccatgtggctcacaccgaagggcaccgtgtgcatcgccaagaaggagatcgtctggt
acccactgattggacagctctacgctctggctggacacctccgcattgacaggtccaaccct
gtggctgctatccagtccatgaaggaagtggcccgcgctgtggtcaagaatgatctctccct
gatcatcttccctgagggcacaaggagcaaggacggccgcctcctgcccttcaagaagggct
tcgtgcacctcgctctgcagaccaggcgcccaattgtgccgatcgtcctgaccggcacacac
atggcttggaggaagggcagcctccacatcaggccaaccccgctgacagtgaagtacctccc
accgatcgtcaccacagactggacacctgatagggtggaggactacaccaagatgatccacg
atatctacgtcaatcacctgcctgagtcccagcagcctctccgccccaaggagagctga
(SEQ ID NO: 16)
MASIGISSLLKNRKLESSFSTGFAKDSFPHSPEKTMIRDDSEHRTIIADGLSVDDDDGWMAV
LISWARLVMCFVLVLITTSIWTLIMVILIPWPCERIKQSNVFGHVSGRMLMWLLGNPVKIEG
AEHANERAIYICNHASPLDIVLTMWLTPKGTVCIAKKEIVWYPLIGQLYALAGHLRIDRSNP
VAAIQSMKEVARAVVKNDLSLIIFPEGTRSKDGRLLPFKKGFVHLALQTRRPIVPIVLTGTH
MAWRKGSLHIRPTPLTVKYLPPIVTTDWTPDRVEDYTKMIHDIYVNHLPESQQPLRPKES

### Example 10—Generation of Transgenic Sorghum Events Expressing Oil Binary Vector

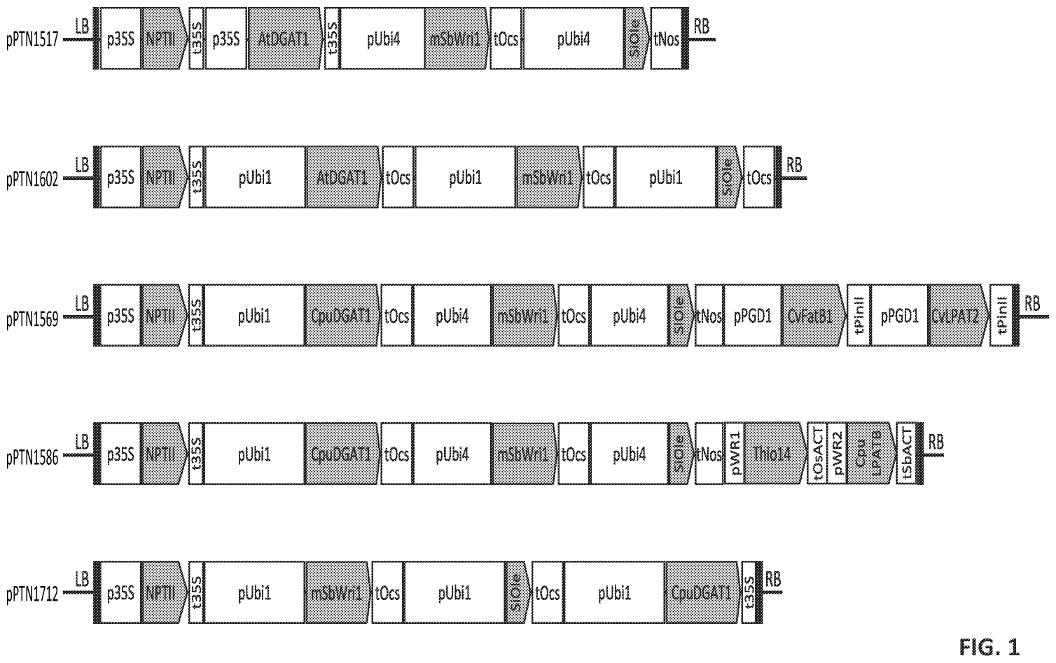

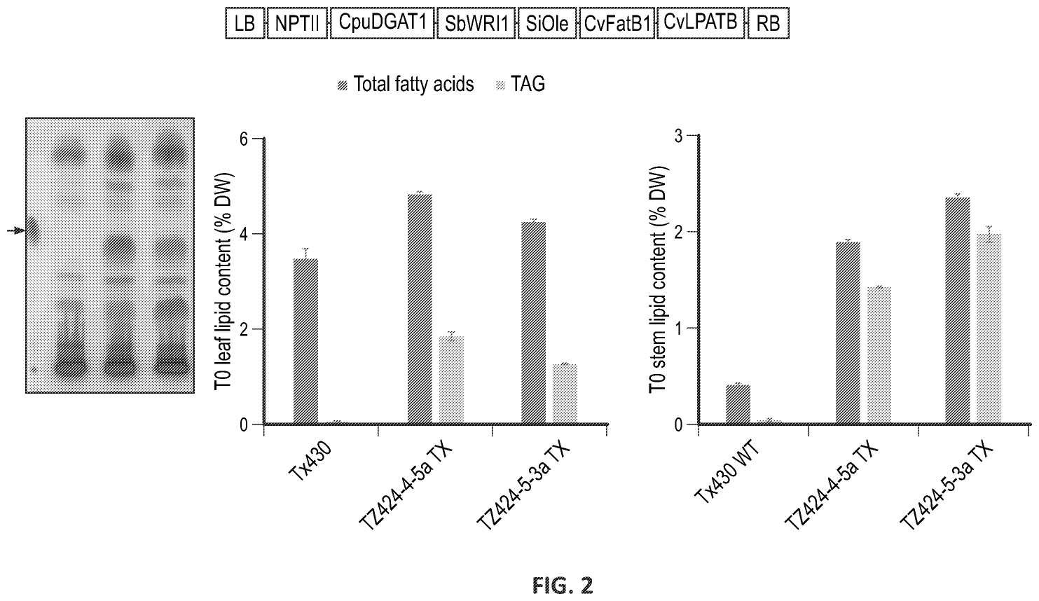

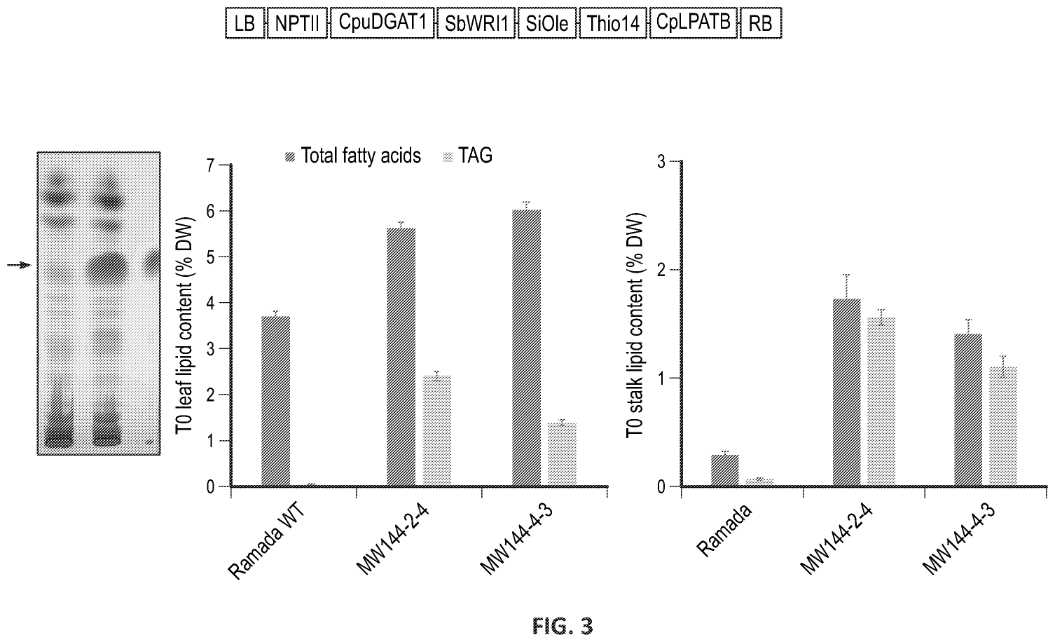

To accumulate oil in vegetative tissues of sorghum, binary vectors containing genes involved in oil biosynthesis and metabolic pathway were constructed via GoldenBraid modular assembly. To achieve the goal, constitutive promoters were used to accelerate the de novo synthesis of fatty acids. First, sorghum Wrinkled1 having one amino mutation, K10R, which is at the residue that participates in protein stability of Arabidopsis Wrinkled1 (Zhai et al., 2017, The Plant Cell, 29:871-89), was introduced. Second, DGAT1 acyltransferase was added to incorporate de novo synthesized free fatty acids into TAG. Finally, sesame oleosin was assembled for protection of TAG by producing oil body. Two different DGAT1 enzymes were used for different approaches; AtDGAT1 for normal TAG accumulation and CpuDGAT1 for medium-chain TAG production. For medium chain TAG, more gene sets were assembled such as thioesterase and LPAT acyltransferase along with three core components. As shown in FIG. 1, pPTN1517 and 1602 are designed to produce normal TAG, while pPTN1569 and 1586 are designed to produce medium-chain TAG. Using Agrobacterium-mediated stable transformation method, transgenic sorghum events harboring these vectors were generated. Phenotype analysis for initial screening was carried out using the T₀ leaf of transgenics (FIGS. 2 and 3). Initial screening results showed that events expressing pPTN1569 (Event name TZ424-5-3a and TZ424-4-5a) or 1586 (Event name MW144-2-4 and MW144-4-3) accumulated oil in their leaves, up to 1.8 and 2.4% DW, respectively, which is ˜50-fold increase compared to wild-type genotypes TX430 and Ramada.

Moreover, events engineered with pPTN1517 or 1602 (i.e., lacking the medium-chain thioesterase) only showed up to ˜6-fold increase of oil in vegetative leaves relative to oil concentrations in leaves of wild=type plants grown and sampled under the same conditions. This result indicated that the addition of the medium-chain thioesterases and Cuphea LPAT provide >8-fold additional oil or TAG above that obtained from the Arabidopsis DGAT, sorghum Wri1 and sesame oleosin transgene combination. Fatty acid profiling of these high oil transgenic plants showed that they unexpectedly accumulated primarily C16 and C18 fatty acids in TAG rather than medium-chain fatty acids in TAG.

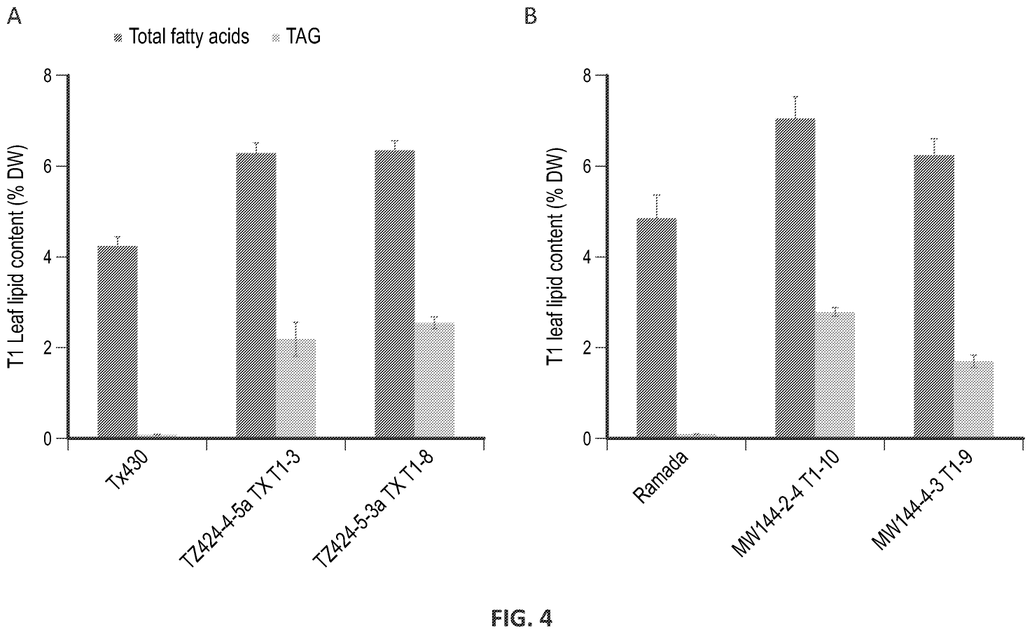

After harvesting T₁ seeds, stem of transgenic events was collected and analyzed. Stalk TAG content was 1.4 and 2.0% DW in TZ424-4-5a and TZ424-5-3a, respectively, and 1.6 and 1.1% DW in MW144-2-4 and MW144-4-3, respectively, which represents a 25 to 40-fold increase in TAG concentrations compared to those in stalks of wild-type (or non-transformed) plants (FIGS. 2 and 3). Total fatty acid concentrations in stems were also increased, including a 4.6- and 5.7-fold increase in TZ424-4-5a and TZ424-5-3a events, respectively, and a 5.9- and 4.8-fold increase in MW144-2-4 and MW 144-4-3 events, respectively, relative to fatty acid concentrations in stems from non-transgenic or wild-type plants grown and harvested under the same conditions. Similar results were obtained from T₁ genotyping-positive progenies; 2.2 to 2.6% DW of TAG in leaves of pPTN1569-engineered events and 1.7 to 2.8% DW of TAG in leaves of pPTN1586-engineered events, which is up to a 35-fold increase in TAG or oil compared to wild-type plants grown and harvested under the same conditions (FIG. 4). To confirm expression of transgenes in transgenic events, droplet digital PCR was performed. In sorghum context, rice PGD1 promoter and synthetic Wrinkled1-inducible promoters (pWR1 and pWR2) showed low, but detectable expression of the introduced thioesterase and LPAT.

### Example 11—Field Trial of Oil Sorghum Event

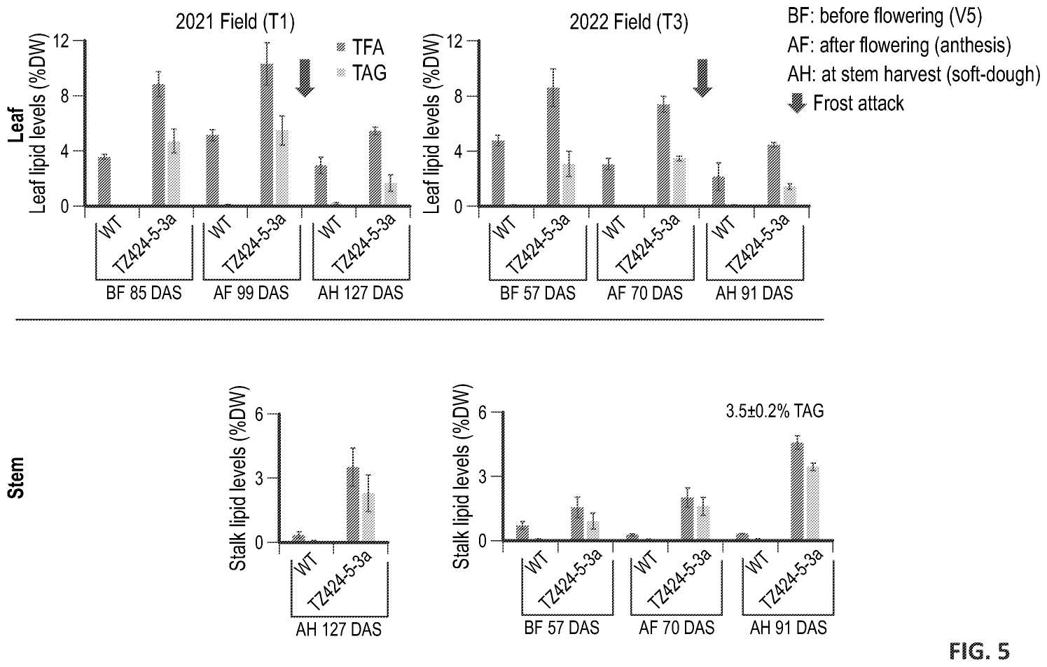

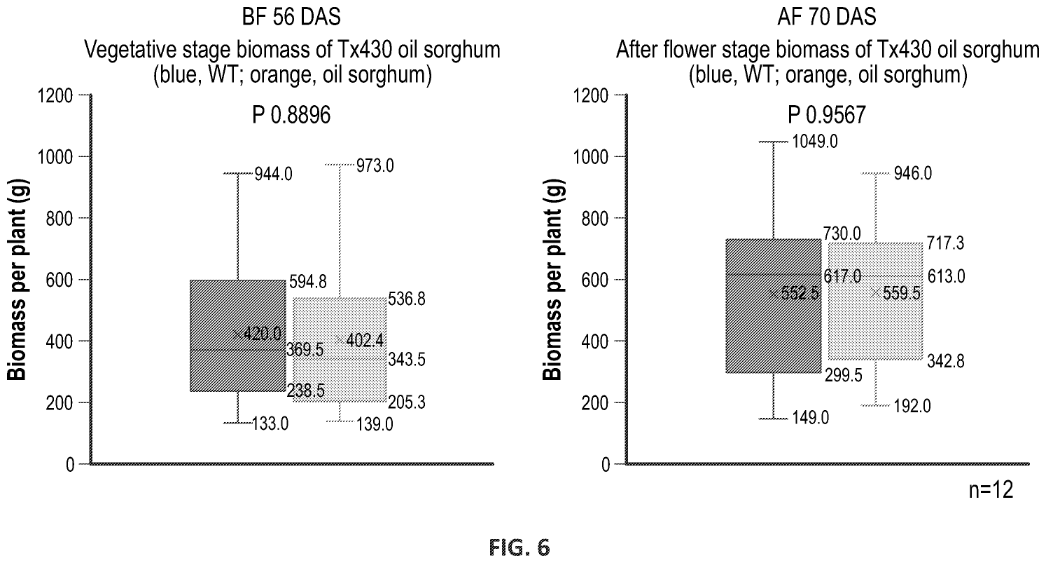

To validate overall agronomic traits of oil sorghum event, TZ424-5-3a event and wild-type were planted in the field at Eastern Nebraska Research and Education Center (ENREC) in the 2021 and 2022 seasons. Total fatty acid and TAG content was measured in three different developmental stages; V5 vegetative stage, anthesis flowering stage, and soft-dough seed filling stage, to determine optimal oil accumulation timing. Two-year field trial results demonstrated that TZ424-5-3a transgenic sorghum event accumulates up to 3.5±0.2% DW of TAG in leaves at 3 months after sowing, which is the growth stage for maximal oil level observed in plants grown under greenhouse conditions (FIG. 5). Total fatty acid concentrations in stems were 4.6+0.3% DW. This is a 56-fold oil increase for TAG and a 14-fold increase for total fatty acid concentrations in stems of transgenic events compared to stems from wild-type control plants grown and harvested under the same conditions. In contrast, after-flowering stage was the optimal timing for leaf TAG accumulation in transgenic sorghum. At this stage, TAG concentrations in leaves of transgenic plants were 5.5 1.0% DW in 2021 and 3.5 0.2% DW in 2022, representing a 70-115-fold increase compared to concentrations in wild-type leaves, while total fatty acid concentrations in leaves of the transgenic plants were 2-fold and 2.4-fold higher than those in leaves of wild-type control plants in 2021 and 2022, respectively. These results indicate that two different vegetative tissues (e.g., leaves and stems) have different oil storing capacity and metabolic mechanisms. Since there is possibility that TAG accumulation in vegetative may affect use of photosynthetic carbon, aerial vegetative parts of plants were harvested and compared between wild-type and transgenic event in two different growth stages (V5 vs anthesis, FIG. 6). Even regarding the high mass variation between the individuals based on multi-tillering trait of TX430 genotype background under the field condition, there was no significant mass difference between wild-type and TZ424-5-3a transgenic sorghum event. These results suggest that under the normal field conditions, one of transgenic sorghum events TZ424-5-3a reaches optimal oil content within 3 months without a biomass yield penalty.

### Example 12—Thioesterase Activity in Sorghum Event

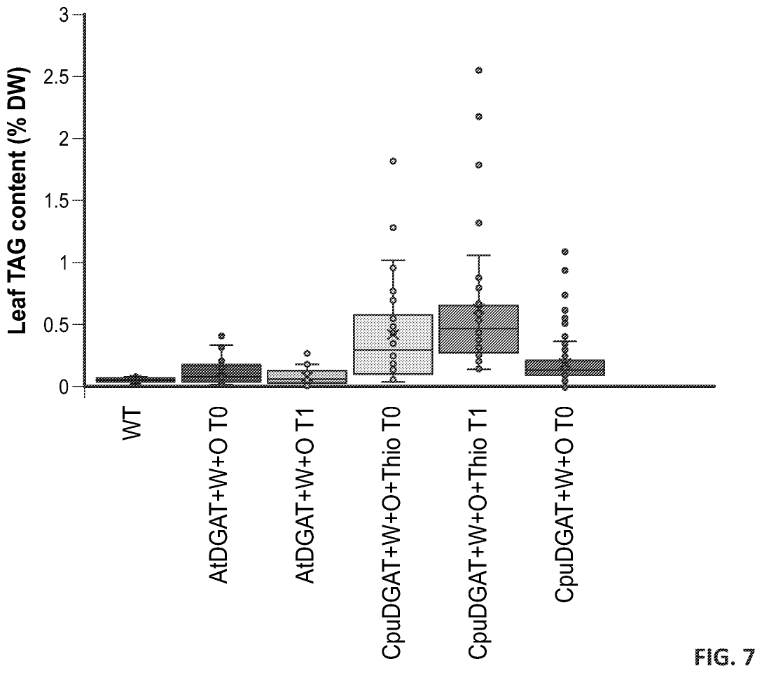

As mentioned above, the transcript of transgene acyl-ACP thioesterase and LPAT was barely detected in transgenic sorghum events harboring pPTN1569 and 1586. We hypothesized that CpuDGAT1 is contributing oil trait in transgenic sorghum events rather than AtDGAT1 from the result of initial screening (FIG. 7), since AtDGAT1-expressing transgenic sorghum events (harboring pPTN1517 or 1602) accumulate no more than 0.5% DW in leaves at both T₀ and T₁ generations, which means ˜10-fold increase compared to wild-type plants.

To test this hypothesis, a new binary vector called pPTN1712 was constructed. This new vector contained CpuDGAT1, mSbWri1, and SiOle under the control of constitutive promoters but no additional gene sets for medium-chain TAG production such as acyl-ACP thioesterase and LPAT. Transgenic events harboring pPTN1712 were generated and analyzed. Initial screening result showed the new vector performed better than pPTN1517 and 1602, but not as well as previous CpuDGATT-containing vector constructs, pPTN1569 and 1586.

To confirm that the addition of the medium-chain acyl-ACP thioesterases and LPAT and not the Cuphea DGAT are responsible for the high fatty acid and TAG phenotypes, new engineered sorghum lines were developed that contained only the three-transgene combination, with Cuphea DGAT rather than the Arabidopsis DGAT. The new lines that lacked the medium-chain acyl-ACP thioesterases and LPAT accumulated total fatty acids and TAG at concentrations similar to those measured in leaves and stems having the three-transgene combination with the Arabidopsis DGAT. These concentrations were two- to five-fold lower than in leaves and stems of sorghum lines engineered with the medium-chain acyl-ACP thioesterase and LPAT plus the three-transgene combination. While medium chain-fatty acids (C8-C14) were not detected in the high oil-engineered lines, elevated medium-chain acyl-ACP thioesterase activity was detected in these lines versus non-transgenic controls, indicating that the introduced thioesterase is active. Without being bound by theory, we propose that the medium-chain fatty acids produced by the added thioesterase are preferentially targeted for catabolism by beta-oxidation. Under this model, this would effectively “tie-up” beta-oxidation and promote the accumulation of longer chain C16 and C18 fatty acids that accumulate in total lipids and TAG of the high oil-engineered lines.

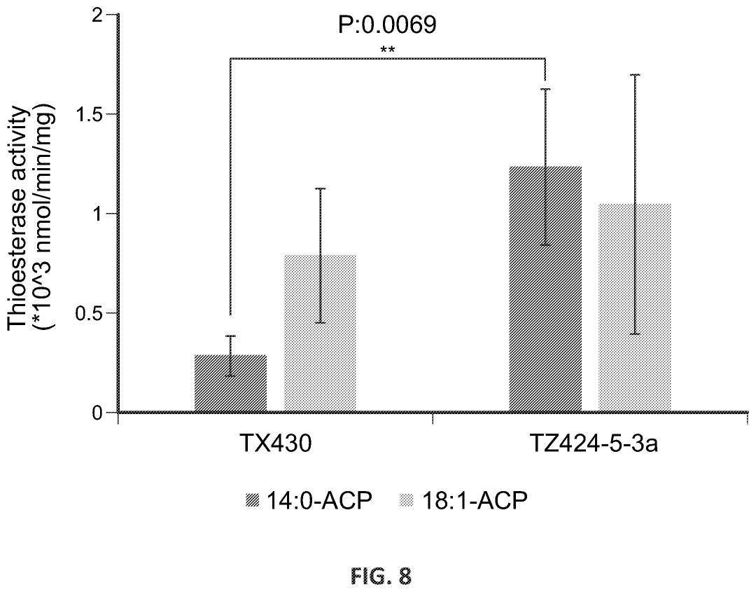

We hypothesized that acyl-ACP thioesterase could play a positive role in oil accumulation even at its low expression level. To test acyl-ACP thioesterase activity of transgenic sorghum, total soluble protein was extracted from 2022 field-grown sorghum leaves and assayed with supplementation of ¹⁴C-labeled substrates, myristoyl-ACP and oleoyl-ACP (FIG. 8). Interestingly, transgenic sorghum event exhibited about 4-fold increased thioesterase activity to myristoyl-ACP, while no activity change was detected to oleoyl-ACP.

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

1. A nucleic acid construct comprising at least one constitutive promoter driving expression of:
a nucleic acid molecule encoding a diacylglycerol acyltransferase 1 (DGAT1) protein;
a nucleic acid molecule encoding a Wrinkled1 (Wri1) protein;
a nucleic acid molecule encoding an oleosin (Ole) protein;
a nucleic acid molecule encoding a medium-chain acyl-ACP thioesterase; and
a nucleic acid molecule encoding a lysophosphatidic acid acyltransferase 2 (LPAT2) protein.

2. The nucleic acid construct of claim 1, wherein the DGAT protein is a Cuphea DGAT 1 protein.

3. The nucleic acid construct of claim 1, wherein the wrinkled1 protein is a Sorghum wrinkled1 protein.

4. The nucleic acid construct of claim 1, wherein the oleosin protein is a sesame oleosin protein.

5. The nucleic acid construct of claim 1, wherein the medium-chain acyl-ACP thioesterase is a FatB protein.

6. The nucleic acid construct of claim 5, wherein the FatB protein is a Cuphea viscosissima FatB protein.

7. The nucleic acid construct of claim 1, wherein the medium-chain acyl-ACP thioesterase protein is a Cuphea palustris medium-chain acyl-ACP thioesterase protein.

8. The nucleic acid construct of claim 1, wherein the LPAT2 protein is a Cuphea viscosissima LPAT2 protein.

9. A nucleic acid construct comprising at least one constitutive promoter driving expression of:
a nucleic acid molecule encoding a Cuphea avigera var. pulcherrima DGAT1 protein;
a nucleic acid molecule encoding a sorghum Wrinkled1 protein;
a nucleic acid molecule encoding a sesame oleosin protein;
a nucleic acid molecule encoding a Cuphea viscosissima FatB protein; and
a nucleic acid molecule encoding a Cuphea viscosissima LPAT2 protein.

10. A nucleic acid construct comprising at least one constitutive promoter driving expression of:
a nucleic acid molecule encoding a Cuphea avigera var. pulcherrima DGAT1 protein;
a nucleic acid molecule encoding a sorghum Wrinkled1 protein;
a nucleic acid molecule encoding a sesame oleosin protein;
a nucleic acid molecule encoding a Cuphea palustris thioesterase protein; and
a nucleic acid molecule encoding a Cuphea avigera var. pulcherrima LPATB protein.

11. The nucleic acid construct of claim 9, wherein the nucleic acid molecule encoding the DGAT1 protein from Cuphea avigera var. pulcherrima has the nucleic acid sequence shown in SEQ ID NO:1.

12. (canceled)

13. The nucleic acid construct of claim 9, wherein the nucleic acid molecule encoding the Wrinkled1 protein from sorghum has the nucleic acid sequence shown in SEQ ID NO:3.

14. (canceled)

15. The nucleic acid construct of claim 9, wherein the nucleic acid molecule encoding the oleosin protein from sesame has the nucleic acid sequence shown in SEQ ID NO:5.

16. (canceled)

17. The nucleic acid construct of claim 9, wherein the nucleic acid molecule encoding the FatB protein from Cuphea viscosissima has the nucleic acid sequence shown in SEQ ID NO:7.

18. (canceled)

19. The nucleic acid construct of claim 9, wherein the nucleic acid molecule encoding the LPAT2 protein from Cuphea viscosissima has the nucleic acid sequence shown in SEQ ID NO:9.

20. (canceled)

21. The nucleic acid construct of claim 10, wherein the nucleic acid molecule encoding the thioesterase protein from Cuphea palustris has the nucleic acid sequence shown in SEQ ID NO:13.

22. (canceled)

23. The nucleic acid construct of claim 10, wherein the nucleic acid molecule encoding the LPATB protein from Cuphea avigera var. pulcherrima has the nucleic acid sequence shown in SEQ ID NO:15.

24. (canceled)

25. The nucleic acid construct of claim 1, wherein the at least one constitutive promoter is selected from the group consisting of a 35S promoter from cauliflower mosaic virus, a ubiquitin 1 promoter from maize, a ubiquitin 4 promoter from sugarcane, and a PGD1 promoter from rice.

26. The nucleic acid construct of claim 1, further comprising more than one constitutive promoter driving expression of the nucleic acid molecules.

27-28. (canceled)

29. The nucleic acid construct of claim 1, further comprising left border nucleic acid sequences and right border nucleic acid sequences.

30. The nucleic acid construct of claim 29, wherein the nucleic acid construct is a T-DNA.

31. A method of making a transgenic plant that produces increased amounts of fatty acids and/or triacylglycerols (TAGs) in vegetative tissues relative to a corresponding plant lacking the construct, the method comprising:
transforming a plant cell with the nucleic acid construct of claim 1, and
regenerating the transformed plant cell into a plant,
thereby making a plant that produces increased amounts of fatty acids and/or triacylglycerols (TAGs) in vegetative tissues relative to a corresponding plant lacking the construct.

32. The method of claim 31, wherein the plant is selected from the group consisting of sorghum, sugarcane, Miscanthus, maize, rye, and switch grass.

33. The method of claim 31, wherein the transforming step is via Agrobacterium transformation.

34. The method of claim 31, wherein the increased amounts of fatty acids in vegetative tissues is about two-fold, three-fold, or four-fold relative to the amount of fatty acids in vegetative tissues in a corresponding plant lacking the construct.

35. A transgenic plant comprising the nucleic acid construct of claim 1.

36. The transgenic plant of claim 35, wherein the plant is selected from the group consisting of sorghum, sugarcane, Miscanthus, maize, rye, and switch grass.

37. The transgenic plant of claim 35, wherein the plant exhibits increased amounts of fatty acids and/or TAGs in vegetative tissues relative to a corresponding plant lacking the construct.


SEQUENCE LISTING (38 sequences)

SEQ ID NO: 1 - DNA, 1482 bp, Cuphea avigera
  source 1..1482: mol_type = other DNA
atgcacgaggccgtgagccacttcctgcacaggcacgctccactcagcctgtccggcttcgccatggctatcgtgtccggcaccctcggagtggctgcttccagcttcatccctgacagcgatcactccaccacaagcccctccctgaggaagcgcaactccagctccctcttcccgaaggccagcgacaccagctccgtggatggcaaggccgctcacaggacaagctccccagtccacctcaagctggctgagtccccgctgagctcccgcaacatcttcaagcagaatcacgagggcctcttcaacctgtgcatggtcacactcgtggccgtcatcatcaggctcttcctggagaatctcctgaagtacggctggctgatgaagcgcgacttctggctcagcaccttcacagcctggcctctgttcatctgctccctgggcctgccaatcttccctctggctgctttcgtggtcgagaagctcgctcagaagaatctcctgcctgagcccatcgtgctgtgcagccacgtcatcatcacatccgcttccgtgctctaccctgctctcgtcatcctgaggttcgactgcgccctgatgagcggcatcggcctcatgctctactcctgcgccctctggctgaagctcgtgagctacgctcacacatcctacgatatgcgctgcgaggctaagagccgcctcgagggcaagagctccgctgactccaagaacggcgagctgccttacagggtgaatatcaaggatctcgcctacttcatggtcgctcctaccctctgctaccagctgagctaccccaggacacagttcatccgcaagttctgggtggcccgccaggtcctgaagctcatcctggtgaacgtggtcatgggcttcatcatcgagcagtacatgatccctgtcatgcacaactccaagccaccgaggcgcggatactggctgcacttcatcgagcgcaatctcaagctggctgtgcccagcatcggcctctggttctgcatcttctactccatcttccacctctggctgaacattgtggctgagctcctgaggttcggcgaccgcgagttctacaaggattggtggaacgctaagaatatggaggagtactggaagatgtggaatatcccagtgcacaggtggatggtccgccacctgtacggcccatgcatgaagaggaagctcccgcgctgggtggccatcagcatctccttcctcctgagcgctgtcctccacgagatctgcgtgtccgtcccatgccacgtgttccagctgtgggccttcaacggcatgatgctccagatcccgctcgtcctgagctccaagcctctgcagaagaggttccccagctccaaggccggcaacgtgttcttctggttcctcttctgcatctacggccagccgaattgcgtcctcatgtactaccatgctctgatggagaggcgcggcctccgcatcgattga

SEQ ID NO: 2 - protein, 493 aa, Cuphea avigera
MHEAVSHFLHRHAPLSLSGFAMAIVSGTLGVAASSFIPDSDHSTTSPSLRKRNSSSLFPKASDTSSVDGKAAHRTSSPVHLKLAESPLSSRNIFKQNHEGLFNLCMVTLVAVIIRLFLENLLKYGWLMKRDFWLSTFTAWPLFICSLGLPIFPLAAFVVEKLAQKNLLPEPIVLCSHVIITSASVLYPALVILRFDCALMSGIGLMLYSCALWLKLVSYAHTSYDMRCEAKSRLEGKSSADSKNGELPYRVNIKDLAYFMVAPTLCYQLSYPRTQFIRKFWVARQVLKLILVNVVMGFIIEQYMIPVMHNSKPPRRGYWLHFIERNLKLAVPSIGLWFCIFYSIFHLWLNIVAELLRFGDREFYKDWWNAKNMEEYWKMWNIPVHRWMVRHLYGPCMKRKLPRWVAISISFLLSAVLHEICVSVPCHVFQLWAFNGMMLQIPLVLSSKPLQKRFPSSKAGNVFFWFLFCIYGQPNCVLMYYHALMERRGLRID

SEQ ID NO: 3 - DNA, 1263 bp, Sorghum bicolor
  source 1..1263: mol_type = genomic DNA
atggatatggagaggagccagcagcagcgctcccctacagagagcccaccgcctccctccccctccagctccagctccagcgtgagcgctgacacagtcctcccaccgcctggcaagaggcgcagggctgctaccacagctaaggccaaggctggcgccaagcctaagcgggctaggaaggatgctgctgctgctgctgacccaccaccaccaccagcggctgctgctgctggcaagaggtccagcgtgtaccgcggcgtcactaggcacaggtggacaggaaggttcgaggctcacctctgggataagcactgcctcgccgctctgcacaacaagaagaagggcaggcaggtgtacctgggcgcctacgatagcgaggaagccgctgctagggcttacgacctcgctgctctgaagtactggggcccagagacactcctgaacttcccggtggaggactactccagcgagatgcccgagatggagggcgtgtcccgggaggagtacctcgccagcctgcgcaggcgctccagcggcttctcccgcggcgtggctaagtacaggggcgtcgctaggcaccaccacaatggccgctgggaggctaggattggaagggtcttcggcaacaagtacctctacctgggcaccttcgatacacaggaagaggccgctaaggcctatgacctcgctgctatcgagtaccgcggcgtgaacgctgtcaccaatttcgatatcagctgctacctcgaccacccactcttcctggcccagctccagcaggagcctcaggtggtcccagctctgaatcaagaggcccagccggaccagtccgagacagagacaatcgcccaggagagcgtgtccagcgaggctaagacaccagacgataacgctgagccggacgataatgccgagcctgacgatatcgctgagcccctgatcaccgtcgacgattccatcgaggagtccctctggagcccgtgcatggattacgagctggacacaatgtccaggagcaacttcggctccagcatcaatctctccgagtggttcaacgacgccgatttcgacagcaatatcggctgcctgttcgatggatgctccgctgtggatgagggcggcaaggatggagtcggcctcgctgatttctccctcctggaggacttctccctgttcgaggctggcgacggccagctcaaggatgtgctgagcgacatggaggaaggcatccagccaccgaccatgatctccgtctgcaactga

SEQ ID NO: 4 - protein, 420 aa, Sorghum bicolor
MDMERSQQQRSPTESPPPPSPSSSSSSVSADTVLPPPGKRRRAATTAKAKAGAKPKRARKDAAAAADPPPPPAAAAAGKRSSVYRGVTRHRWTGRFEAHLWDKHCLAALHNKKKGRQVYLGAYDSEEAAARAYDLAALKYWGPETLLNFPVEDYSSEMPEMEGVSREEYLASLRRRSSGFSRGVAKYRGVARHHHNGRWEARIGRVFGNKYLYLGTFDTQEEAAKAYDLAAIEYRGVNAVTNFDISCYLDHPLFLAQLQQEPQVVPALNQEAQPDQSETETIAQESVSSEAKTPDDNAEPDDNAEPDDIAEPLITVDDSIEESLWSPCMDYELDTMSRSNFGSSINLSEWFNDADFDSNIGCLFDGCSAVDEGGKDGVGLADFSLLEDFSLFEAGDGQLKDVLSDMEEGIQPPTMISVCN

SEQ ID NO: 5 - DNA, 438 bp, Sesamum indicum
  source 1..438: mol_type = genomic DNA
atggcgtgtcattatggtcaacaacaacaaacctgtgctccccacctccaactccaacctcgggcgtgtagggtcgtgaaggcagccaccgcagtcacagcaggaggctccctcctcgtgctctcaggtcttaccttggccggaacagttatcgcgctgacgattgcaactccactcctcgtgatcttctcccctgtgcttgtccccgctgtcatcacgattttcctcctgggcgccgggtttctcgcttcaggtggattcggcgttgccgctctctccgtgctgagctggatctacaggtatctgacgggcaagcacccaccgggggcagactgccttgagtcggcaaagactaagttggcctcctgtgctcgcgagatgaaggatagagcggaacagttttcttgtcaaccagtcgcaggtagccagaccagctag

SEQ ID NO: 6 - protein, 145 aa, Sesamum indicum
MACHYGQQQQTCAPHLQLQPRACRVVKAATAVTAGGSLLVLSGLTLAGTVIALTIATPLLVIFSPVLVPAVITIFLLGAGFLASGGFGVAALSVLSWIYRYLTGKHPPGADCLESAKTKLASCAREMKDRAEQFSCQPVAGSQTS

SEQ ID NO: 7 - DNA, 1260 bp, Cuphea viscosissima
  source 1..1260: mol_type = genomic DNA
atggtggccgctgccgctacatccgctttcttccctgtcccagcgcctggcacaagccctaagcctggcaagtccggaaactggccttccagcctcagcccaaccttcaagccgaagtccatccctaatggcggcttccaggtcaaggccaacgctagcgctcaccctaaggctaacggctccgctgtgaatctcaagtccggctccctgaatacccaagaggacacatccagctccccaccgcctcgcgctttcctcaaccagctgcccgattggagcatgctcctgaccgccatcaccacagtgttcgtcgccgctgagaagcagtggacaatgctcgacaggaagtccaagcgcccagacatgctcgtggatagcgtcggcctgaagtccattgtgagggacggcctggtcagcaggcacagcttctccatcaggtcctacgagatcggcgctgataggaccgccagcatcgagacactcatgaatcacctgcaggagacaacaatcaaccactgcaagtccctcggcctgcataatgatggattcggaaggacccctggaatgtgcaagaacgatctcatctgggtgctgacaaagatgcagatcatggtcaataggtaccctacctggggcgacacagtcgagatcaacacctggttcagccagtccggcaagatcggcatggccagcgactggctcatctccgattgcaataccggcgagatcctgatccgcgctacaagcgtctgggccatgatgaaccagaagaccaggcgcttctcccgcctcccatacgaggtgaggcaggagctgacaccccacttcgtggatagcccacacgtcatcgaggacaatgatcagaagctgaggaagttcgacgtcaagaccggcgattccatccgcaagggcctcacacccaggtggaacgacctggatgtgaatcagcacgtgagcaacgtcaagtacatcggctggatcctcgagtccatgccaatcgaggtgctggagacacaggagctctgctccctgacagtcgagtacaggcgcgagtgcggcatggacagcgtgctcgagtccgtgacagctgtggatccttccgagaacggcggccgctcccagtacaagcacctcctgaggctggaggacggcaccgatatcgtcaagagcaggacagagtggcgccctaagaacgctggcaccaatggcgccatcagcacctccacagccaagacatccaacggcaatagcgtgtcctga

SEQ ID NO: 8 - protein, 419 aa, Cuphea viscosissima
MVAAAATSAFFPVPAPGTSPKPGKSGNWPSSLSPTFKPKSIPNGGFQVKANASAHPKANGSAVNLKSGSLNTQEDTSSSPPPRAFLNQLPDWSMLLTAITTVFVAAEKQWTMLDRKSKRPDMLVDSVGLKSIVRDGLVSRHSFSIRSYEIGADRTASIETLMNHLQETTINHCKSLGLHNDGFGRTPGMCKNDLIWVLTKMQIMVNRYPTWGDTVEINTWFSQSGKIGMASDWLISDCNTGEILIRATSVWAMMNQKTRRFSRLPYEVRQELTPHFVDSPHVIEDNDQKLRKFDVKTGDSIRKGLTPRWNDLDVNQHVSNVKYIGWILESMPIEVLETQELCSLTVEYRRECGMDSVLESVTAVDPSENGGRSQYKHLLRLEDGTDIVKSRTEWRPKNAGTNGAISTSTAKTSNGNSVS

SEQ ID NO: 9 - DNA, 1155 bp, Cuphea viscosissima
  source 1..1155: mol_type = genomic DNA
atggccatcgccgctgccgctgtcatcttcctgttcggcctcatcttcttcgcctccggcctgatcatcaacctcttccaggctctgtgcttcgtcctcatcaggccgctgtccaagaacgcctacaggcgcatcaatcgcgtgttcgctgagctcctgctcagcgagctgctgtgcctcttcgactggtgggctggagctaagctgaagctcttcaccgatcctgagacattcaggctcatgggcaaggagcacgccctcgtgatcatcaaccacatgaccgagctcgactggatggtcggctgggtcatgggccagcacttcggctgcctgggctccatcatcagcgtcgccaagaagtccacaaagttcctgccggtgctcggctggtccatgtggttcagcgagtacctgtacctcgagaggagctgggctaaggacaagtccaccctcaagagccacatcgagcgcctgatcgattaccccctcccattctggctggtcatcttcgtggagggcacaaggttcacccgcacaaagctgctggctgctcagcagtacgccgtgtcctccggcctccctgtccctaggaatgtgctgatcccccgcaccaagggcttcgtgtcctgcgtgagccacatgcgctccttcgtccccgccgtgtacgacgtcacagtggctttcccaaagacctccccaccgcctacactgctcaatctcttcgagggccagagcatcatgctgcacgtccacatcaagaggcacgccatgaaggacctgccagagagcgatgatgctgtggctgagtggtgcagggataagttcgtggagaaggacgccctgctcgataagcacaacgctgaggataccttctccggccaagaggtgaggcacacaggctcccgccagctcaagagcctgctcgtggtcatcagctgggtggtcgtgaccacattcggcgctctgaagttcctccagtggtccagctggaagggcaaggccttctccgctatcggcctgggcatcgtcaccctgctcatgcacgtgctgatcctctccagccaggccgagaggagcaaccctgctgaggtcgcccaggctaagctcaagaccggcctgtccatcagcaagaaggtgacagataaggagaattga

SEQ ID NO: 10 - protein, 384 aa, Cuphea viscosissima
MAIAAAAVIFLFGLIFFASGLIINLFQALCFVLIRPLSKNAYRRINRVFAELLLSELLCLFDWWAGAKLKLFTDPETFRLMGKEHALVIINHMTELDWMVGWVMGQHFGCLGSIISVAKKSTKFLPVLGWSMWFSEYLYLERSWAKDKSTLKSHIERLIDYPLPFWLVIFVEGTRFTRTKLLAAQQYAVSSGLPVPRNVLIPRTKGFVSCVSHMRSFVPAVYDVTVAFPKTSPPPTLLNLFEGQSIMLHVHIKRHAMKDLPESDDAVAEWCRDKFVEKDALLDKHNAEDTFSGQEVRHTGSRQLKSLLVVISWVVVTTFGALKFLQWSSWKGKAFSAIGLGIVTLLMHVLILSSQAERSNPAEVAQAKLKTGLSISKKVTDKEN

SEQ ID NO: 11 - DNA, 1563 bp, Arabidopsis thaliana
  source 1..1563: mol_type = genomic DNA
atggcgattctggattcggctggggtgactactgtgacggaaaacggggggggcgagttcgtggacctggataggctgcggcggcggaagagcaggtctgattccagcaacggactcctgctttcaggctcggacaacaattccccaagcgacgatgtgggagcaccagctgacgtccgggatagaatcgacagcgtggtcaacgacgatgcccaagggacggccaatctggctggcgacaacaatggcggaggtgacaacaacggcggaggaaggggtggaggagagggaaggggtaacgcagatgcaaccttcacatacaggccatccgtgccagcacacaggagagcacgcgaaagccctctctcttcagacgccatctttaagcagtcgcatgctggattgttcaatctctgcgttgtggtcttgattgccgtcaactctcggttgatcattgagaatctcatgaagtacggctggcttatcagaacggatttctggttttcgtccaggtccctgcgcgactggcctcttttcatgtgctgtatctctttgtcaattttccccctcgccgcttttactgtcgagaagttggttctccaaaagtatatcagcgaaccagttgtgatttttctgcacatcattatcacgatgactgaggtcttgtacccggtctatgttaccctccggtgcgattcagcgttcctgtcgggcgttacccttatgttgctcacatgtatcgtttggctgaagcttgtgtcttacgcacatacatcatatgatattaggtcgctcgcgaacgcggcagacaaggcaaatccagaggtgtcctactatgtcagcctgaagtctcttgcctacttcatggtggctccgaccctgtgctaccagccttcctatccccggagcgcctgtatcagaaaggggtgggttgccaggcaattcgctaagctcgtgatttttacaggattcatgggctttattatcgagcagtacatcaacccaattgtcaggaatagcaagcacccgctgaagggcgacctcctttatgctatcgaacgcgtcttgaagctctccgttcctaacctctacgtgtggctttgcatgttctactgtttctttcatctgtggcttaacatcctcgccgagttgctctgcttcggagatcgggaattttacaaggactggtggaacgctaagtccgtgggcgactattggagaatgtggaatatgcccgtccacaagtggatggttcggcatatctacttcccttgcctgagatctaagattcccaagaccctggcgattatcattgcatttcttgtgtcagcggtcttccacgagctgtgcatcgcagtcccttgtaggttgttcaagctctgggcgtttcttggcatcatgttccaggttcccttggtgtttattacgaactacctccaagaacgcttcgggtccactgtgggtaatatgatcttctggtttatcttctgcattttcgggcagccgatgtgtgtcctgctttactatcatgacctcatgaaccgcaagggttcgatgtcctga

SEQ ID NO: 12 - protein, 520 aa, Arabidopsis thaliana
MAILDSAGVTTVTENGGGEFVDLDRLRRRKSRSDSSNGLLLSGSDNNSPSDDVGAPADVRDRIDSVVNDDAQGTANLAGDNNGGGDNNGGGRGGGEGRGNADATFTYRPSVPAHRRARESPLSSDAIFKQSHAGLFNLCVVVLIAVNSRLIIENLMKYGWLIRTDFWFSSRSLRDWPLFMCCISLSIFPLAAFTVEKLVLQKYISEPVVIFLHIIITMTEVLYPVYVTLRCDSAFLSGVTLMLLTCIVWLKLVSYAHTSYDIRSLANAADKANPEVSYYVSLKSLAYFMVAPTLCYQPSYPRSACIRKGWVARQFAKLVIFTGFMGFIIEQYINPIVRNSKHPLKGDLLYAIERVLKLSVPNLYVWLCMFYCFFHLWLNILAELLCFGDREFYKDWWNAKSVGDYWRMWNMPVHKWMVRHIYFPCLRSKIPKTLAIIIAFLVSAVFHELCIAVPCRLFKLWAFLGIMFQVPLVFITNYLQERFGSTVGNMIFWFIFCIFGQPMCVLLYYHDLMNRKGSMS

SEQ ID NO: 13 - DNA, 1236 bp, Cuphea palustris
  source 1..1236: mol_type = genomic DNA
atggttgcagctgctgcaagtgccgcattcttttctgtagctacaccgagaactaatatctctccatcatccctcagtgttccgtttaagccaaagagtaatcataacggcgggttccaagttaaggcgaacgcttcagctcatccgaaggcaaacgggagtgcagtttccctcaaatcaggctctcttgagactcaggaggacaagacaagctcaagtagtccacctcctcgaactttcattaatcagcttcctgtttggtcaatgttactttcagctgtcacaactgtttttggcgtggctgagaagcagtggcctatgttggatcgtaaaagtaagaggccagacatgttagttgagcctctgggagttgatagaattgtttatgatggagtttcattccgtcaatcgttcagcattcgatcctacgaaataggagccgacagaactgcttcaattgaaacccttatgaatatgtttcaagaaacttcactaaaccactgtaaaattataggacttcttaatgatggcttcggaaggactcctgaaatgtgtaagagagatttgatatgggttgttactaagatgcaaatcgaggtgaacagatatccgacttggggagatactattgaggttaacacttgggtttctgcaagcggcaaacatggtatgggacgagattggcttatctcagattgtcacacaggtgagatattgattagggccacgtctgtttgggcaatgatgaaccaaaagactagaaggctatcaaagataccttacgaagttaggcaagaaatagaaccgcagtttgtagattcagcgccagttatcgttgatgacagaaagtttcataaacttgatcttaaaactggagattccatttgtaacggtcttactccgaggtggacagatttggatgtgaaccagcacgtaaacaacgttaaatatatcgggtggatactccagtctgttcctactgaggttttcgagactcaggagttatgtggactcacgcttgagtacagaagagagtgcggacgtgactccgttctggagagcgttactgctatggacccttcaaaagaaggggatcgttccttgtaccaacatcttcttcgtttagaagatggagcagatatcgtgaaagggagaaccgagtggcgtccaaaaaatgcgggagcaaagggagccattcttacaggaaagacaagcaacggaaattcaatctcttga

SEQ ID NO: 14 - protein, 411 aa, Cuphea palustris
MVAAAASAAFFSVATPRTNISPSSLSVPFKPKSNHNGGFQVKANASAHPKANGSAVSLKSGSLETQEDKTSSSSPPPRTFINQLPVWSMLLSAVTTVFGVAEKQWPMLDRKSKRPDMLVEPLGVDRIVYDGVSFRQSFSIRSYEIGADRTASIETLMNMFQETSLNHCKIIGLLNDGFGRTPEMCKRDLIWVVTKMQIEVNRYPTWGDTIEVNTWVSASGKHGMGRDWLISDCHTGEILIRATSVWAMMNQKTRRLSKIPYEVRQEIEPQFVDSAPVIVDDRKFHKLDLKTGDSICNGLTPRWTDLDVNQHVNNVKYIGWILQSVPTEVFETQELCGLTLEYRRECGRDSVLESVTAMDPSKEGDRSLYQHLLRLEDGADIVKGRTEWRPKNAGAKGAILTGKTSNGNSIS

SEQ ID NO: 15 - DNA, 927 bp, Cuphea avigera
  source 1..927: mol_type = genomic DNA
atggcctccatcggcatctccagcctcctgaagaacaggaagctggagtccagcttcagcacaggcttcgctaaggattccttccctcacagccccgagaagacaatgatcagggacgattccgagcacaggaccatcatcgctgatggcctcagcgtggacgatgacgatggctggatggccgtgctgatctcctgggctcgcctcgtcatgtgcttcgtgctcgtcctgatcaccacaagcatctggaccctgatcatggtcatcctcatcccttggccctgcgagaggatcaagcagtccaacgtgttcggccacgtcagcggccgcatgctgatgtggctcctgggcaatcctgtgaagattgagggagctgagcatgctaacgagagggccatctacatctgcaatcacgcttccccactcgatatcgtcctgaccatgtggctcacaccgaagggcaccgtgtgcatcgccaagaaggagatcgtctggtacccactgattggacagctctacgctctggctggacacctccgcattgacaggtccaaccctgtggctgctatccagtccatgaaggaagtggcccgcgctgtggtcaagaatgatctctccctgatcatcttccctgagggcacaaggagcaaggacggccgcctcctgcccttcaagaagggcttcgtgcacctcgctctgcagaccaggcgcccaattgtgccgatcgtcctgaccggcacacacatggcttggaggaagggcagcctccacatcaggccaaccccgctgacagtgaagtacctcccaccgatcgtcaccacagactggacacctgatagggtggaggactacaccaagatgatccacgatatctacgtcaatcacctgcctgagtcccagcagcctctccgccccaaggagagctga

SEQ ID NO: 16 - protein, 308 aa, Cuphea avigera
MASIGISSLLKNRKLESSFSTGFAKDSFPHSPEKTMIRDDSEHRTIIADGLSVDDDDGWMAVLISWARLVMCFVLVLITTSIWTLIMVILIPWPCERIKQSNVFGHVSGRMLMWLLGNPVKIEGAEHANERAIYICNHASPLDIVLTMWLTPKGTVCIAKKEIVWYPLIGQLYALAGHLRIDRSNPVAAIQSMKEVARAVVKNDLSLIIFPEGTRSKDGRLLPFKKGFVHLALQTRRPIVPIVLTGTHMAWRKGSLHIRPTPLTVKYLPPIVTTDWTPDRVEDYTKMIHDIYVNHLPESQQPLRPKES

SEQ ID NO: 17 - DNA, 21 bp, synthetic construct
  source 1..21: mol_type = other DNA
caactttgtcacccgcgatga

SEQ ID NO: 18 - DNA, 21 bp, synthetic construct
  source 1..21: mol_type = other DNA
tccagaaaccttagcagccca

SEQ ID NO: 19 - DNA, 20 bp, synthetic construct
  source 1..20: mol_type = other DNA
aagccgctgctagggcttac

SEQ ID NO: 20 - DNA, 20 bp, synthetic construct
  source 1..20: mol_type = other DNA
gattgatgctggagccgaag

SEQ ID NO: 21 - DNA, 20 bp, synthetic construct
  source 1..20: mol_type = other DNA
gcgaggctaagacaccagac

SEQ ID NO: 22 - DNA, 20 bp, synthetic construct
  source 1..20: mol_type = other DNA
gccgatattgctgtcgaaat

SEQ ID NO: 23 - DNA, 20 bp, synthetic construct
  source 1..20: mol_type = other DNA
ggcagacaaggcaaatccag

SEQ ID NO: 24 - DNA, 20 bp, synthetic construct
  source 1..20: mol_type = other DNA
cctacaagggactgcgatgc

SEQ ID NO: 25 - DNA, 21 bp, synthetic construct
  source 1..21: mol_type = other DNA
tgggttgatgtactgctcgat

SEQ ID NO: 26 - DNA, 20 bp, synthetic construct
  source 1..20: mol_type = other DNA
cccacctccaactccaacct

SEQ ID NO: 27 - DNA, 20 bp, synthetic construct
  source 1..20: mol_type = other DNA
ggtctggctacctgcgactg

SEQ ID NO: 28 - DNA, 20 bp, synthetic construct
  source 1..20: mol_type = other DNA
ccaggaggaaaatcgtgatg

SEQ ID NO: 29 - DNA, 20 bp, synthetic construct
  source 1..20: mol_type = other DNA
atcctggtgaacgtggtcat

SEQ ID NO: 30 - DNA, 20 bp, synthetic construct
  source 1..20: mol_type = other DNA
gagctcagccacaatgttca

SEQ ID NO: 31 - DNA, 20 bp, synthetic construct
  source 1..20: mol_type = other DNA
aacacctggttcagccagtc

SEQ ID NO: 32 - DNA, 20 bp, synthetic construct
  source 1..20: mol_type = other DNA
gtgtgggctatccacgaagt

SEQ ID NO: 33 - DNA, 20 bp, synthetic construct
  source 1..20: mol_type = other DNA
ctgggcaatcctgtgaagat

SEQ ID NO: 34 - DNA, 20 bp, synthetic construct
  source 1..20: mol_type = other DNA
agccagagcgtagagctgtc

SEQ ID NO: 35 - DNA, 20 bp, synthetic construct
  source 1..20: mol_type = other DNA
tcggaaggactcctgaaatg

SEQ ID NO: 36 - DNA, 20 bp, synthetic construct
  source 1..20: mol_type = other DNA
caaacagacgtggccctaat

SEQ ID NO: 37 - DNA, 20 bp, synthetic construct
  source 1..20: mol_type = other DNA
ctgggcaatcctgtgaagat

SEQ ID NO: 38 - DNA, 20 bp, synthetic construct
  source 1..20: mol_type = other DNA
agccagagcgtagagctgtc